SEQUENCE LISTING (4 sequences)

SEQ ID NO: 1 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 231 aa, Mus sp
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLPLTLVLLAAAWGLRWQRARRRGELHPGVPLPSHP

SEQ ID NO: 3 - protein, 8 aa, Artificial Sequence
  Chicken Egg Ovalbumin
SIINFEKL

SEQ ID NO: 4 - protein, 17 aa, Artificial Sequence
  Chicken Egg Ovalbumin
ISQAVHAAHAEINEAGR

(19) United States
(12) Patent Application Publication
(10) Pub. No.: US 2004/0022760 A1
(43) Pub. Date: Feb. 5, 2004

(54) Methods of using Flt3-ligand in immunization protocols

(76) Inventors: Hilary J. McKenna, Seattle, WA (US); David N. Liebowitz, Seattle, WA (US); Charles R. Maliszewski, Seattle, WA (US)

Correspondence Address: IMMUNEX CORPORATION, LAW DEPARTMENT, 51 UNIVERSITY STREET, SEATTLE, WA, 98101

(21) Appl. No.: 10/401,364

(22) Filed: Mar. 26, 2003

Related U.S. Application Data
(60) Provisional application No. 60/368,263, filed on Mar. 26, 2002.
(60) Provisional application No. 60/427,835, filed on Nov. 19, 2002.

(51) Int. Cl. A61K 39/00; A61K 38/21; A61K 38/19; A61K 38/20

(52) U.S. Cl. 424/85.1; 424/85.7; 424/185.1; 424/85.2

(57) ABSTRACT

The present invention relates to methods of using Flt3-ligand (Flt3-L) in immunization protocols to enhance immune responses against vaccine antigens. Embodiments include administering Flt3-ligand prior to immunizing a subject with a vaccine, wherein the vaccine comprises at least one antigen formulated in one or more adjuvants. Methods of treating and preventing disease and infection using Flt3-ligand immunization protocols are also provided. Methods of using Flt3-ligand immunization protocols for in vivo evaluation of antigens and adjuvants are also provided.

### RELATED APPLICATIONS

This application hereby claims the benefit of U.S. provisional application serial No. 60/427,835, filed Nov. 19, 2002, and U.S. provisional application serial No. 60/368,263, filed Mar. 26, 2002, the disclosure of which is relied upon and incorporated by reference.

### FIELD OF THE INVENTION

The present invention relates to methods of using Flt3-ligand in immunization protocols to enhance immune responses against antigens. Embodiments include administering Flt3-ligand prior to immunizing a subject with a vaccine, wherein the vaccine comprises an antigen and an adjuvant. Methods of treating and preventing disease and infection using Flt3-ligand immunization protocols are provided. Methods of using Flt3-ligand immunization protocols for in vivo evaluation of antigens and adjuvants are also provided.

### BACKGROUND OF THE INVENTION

The objective of vaccination is to provide effective immunity by establishing adequate levels of antigen-specific antibodies and a primed population of effector cells that can rapidly expand on renewed contact with antigen. Vaccination is an efficient means of preventing death or disability from infectious diseases and numerous vaccines are licensed for administration to humans, including live virus vaccines for certain adenoviruses, measles, mumps and rubella viruses, and poliovirus, diphtheria and tetanus toxoid vaccines, and Haemophilus b and meningococcal polysaccharide vaccines (Hinman et al., in Principles and Practice of Infectious Diseases, 3rd edition; G. L. Mandell, R. G. Douglas and J. E. Bennett, eds., Churchill Livingstone Inc., NY, N.Y.; 2320-2333; Table 2). The success of vaccination in treating and preventing infectious disease has stimulated interest in utilizing vaccines in the treatment or prevention of neoplastic disease. With the realization in the art that tumors can express tumor-specific antigens, immunization strategies have been developed that utilize MHC class I-restricted peptides derived from tumor antigens. However, clinical trials have had limited success and it is clear that the immunization strategies have not been optimal.

A frequent difficulty with immunization protocols is that the vaccine antigen does not possess sufficient immunogenicity to promote a strong immune response, and therefore a sufficient level of protection against subsequent challenge by the same antigen. In addition, certain antigens may elicit only weak cell-mediated or antibody responses. Depending on the particular disease, a strong cell-mediated and/or humoral immune response may be desirable. For decades, researchers have experimented with diverse compounds to increase the immunogenicity of vaccines. Adjuvants are substances that enhance, augment or potentiate an immune response, and can in some instances be used to promote one type of immune response over another. In addition, the relatively weak immunogenicity of certain novel recombinant antigens has required adjuvants to be more potent. Vaccine adjuvants have different modes of action, affecting the immune response both quantitatively and qualitatively. Such modes of action include mobilizing T cells, acting as depots and altering lymphocyte circulation so that these cells remain localized in draining lymph nodes. They may also serve to focus antigen at the site of immunization, thereby allowing antigen specific T cells and B cells to interact more efficiently with antigen-presenting cells. They may also stimulate proliferation and differentiation of T cells and have effects on B cells, such as enhancing the production of different Ig isotypes. Adjuvants may also stimulate and affect the behavior of antigen-presenting cells, particularly dendritic cells and macrophages, rendering them more effective for presenting antigen to T cells and B cells. Unfortunately, few adjuvants have been approved for use in humans and those that have received FDA approval are comparatively weak in their immunopotentiating effect.

Despite the advances in antigen delivery and vaccinology, there are still many challenges in developing efficacious vaccine therapies for cancer and infectious disease. The embodiments described herein address these needs.

### SUMMARY OF THE INVENTION

Embodiments of the invention are drawn to Flt3-ligand immunization protocols and methods of using the same.

In one embodiment, Flt3-ligand immunization protocols relate to a method of immunizing a subject, comprising the steps of:

(a) administering Flt3-ligand to a subject;

(b) optionally administering an auxiliary molecule; and,

(c) administering a vaccine to the subject, wherein the vaccine comprises an antigen and an adjuvant, and wherein Flt3-ligand is administered prior to, concurrent with and/or subsequent to administration of the vaccine, and wherein the auxiliary molecule is administered prior to, concurrent with and/or subsequent to administration of the vaccine, and wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

Additional embodiments are drawn to methods of enhancing an immune response in a subject to a vaccine antigen.

Other embodiments are drawn to methods of increasing the number of antigen-specific effector cells in a subject.

Further embodiments are drawn to methods of enhancing the number of antigen-specific CD8+ cytotoxic T cells and/or CD4+ T-helper cells in a subject.

Additional embodiments are drawn to preventing and/or treating cancer using a Flt3-ligand immunization protocol.

In other aspects, embodiments are drawn to preventing and/or treating viral infections using a Flt3-ligand immunization protocol.

Further embodiments are drawn to preventing and/or treating bacterial infections using a Flt3-ligand immunization protocol.

Additional embodiments are drawn to preventing and/or treating infection by unicellular organisms using a Flt3-ligand immunization protocol.

In still other embodiments, Flt3-ligand immunization protocols may be used in animal models or test subjects to test the efficacy of vaccines, immunogenicity of antigens or for evaluating protective immune responses to an antigen and/or vaccine in a challenge model.

Further embodiments are drawn to treating allergy patients comprising administering Flt3-ligand in combination with allergen-specific immunotherapy.

### BRIEF DESCRIPTION OF THE DRAWINGS

FIGS. 1A-E represent data derived from FACS (Fluorescence-Activated Cell Sorting) analysis and cell counts of samples isolated from the draining lymph nodes taken at day 1 post immunization of a Flt3-ligand immunization protocol addressing whether Flt3-ligand, pegylated GM-CSF and CD40L enhance immune responses to vaccines (see Example 8). FIG. 1A: total number of cells isolated from the draining lymph nodes; FIG. 1B: percent OT-II transgenic CD4+ T-cells; FIG. 1C: absolute number of OT-II transgenic CD4+ T-cells; FIG. 1D: percent OT-I transgenic CD8+ T-cells; and FIG. 1E: absolute number of OT-I transgenic CD8+ T-cells.

FIGS. 2A-E represent data derived from FACS analysis and cell counts of samples isolated from the draining lymph nodes taken at day 5 post immunization of a Flt3-ligand immunization protocol addressing whether Flt3-ligand, pegylated GM-CSF and CD40L enhance immune responses to vaccines (see Example 8). FIG. 2A: total number of cells isolated from the draining lymph nodes; FIG. 2B: percent OT-I transgenic CD8+ T-cells; FIG. 2C: absolute number of OT-I transgenic CD8+ T-cells; FIG. 2D: percent CD8+ T-cells from host animal; and FIG. 2E: absolute number of CD8+ T-cells from host animal.

FIGS. 3A-E represent data derived from FACS analysis and cell counts of samples isolated from the draining lymph nodes taken at day 9 post immunization of a Flt3-ligand immunization protocol addressing whether Flt3-ligand, pegylated GM-CSF and CD40L enhance immune responses to vaccines (see Example 8). FIG. 3A: total number of cells isolated from the draining lymph nodes; FIG. 3B: percent OT-I transgenic CD8+ T-cells; FIG. 3C: absolute number of OT-I transgenic CD8+ T-cells.; FIG. 3D: percent CD8+ T-cells from host animal; and FIG. 3E: absolute number of CD8+ T-cells from host animal.

FIGS. 4A-B represent standard CTL assays of OT-I transgenic CD8+ T-cells isolated at days 1 (FIG. 4A) and 5 (FIG. 4B) post immunization from the Flt3-ligand immunization protocol described in Example 8. The transgenic CD8+ T-cells were restimulated in vitro prior to inclusion in the CTL assay by culturing with OT-I peptide-pulsed targets.

FIGS. 5A-B represent “ex vivo” CTL assays of OT-I transgenic CD8+ T-cells isolated at days 5 (FIG. 5A) and 9 (FIG. 5B) post immunization from the Flt3-ligand immunization protocol described in Example 8. The transgenic CD8+ T-cells were used directly in the CTL assay without in vitro expansion.

FIGS. 6A-C represent data derived from FACS analysis and cell counts of samples isolated from the draining lymph nodes taken at day 5 post immunization of a Flt3-ligand immunization protocol described in Example 9. FIG. 6A: absolute number of cells isolated from the draining lymph nodes; FIG. 6B: percent OT-I transgenic CD8+ T-cells; and FIG. 6C: absolute number of OT-I transgenic CD8+ T-cells.

FIGS. 7A-C represent data derived from FACS analysis and cell counts of samples isolated from the draining lymph nodes taken at day 5 post immunization of a Flt3-ligand immunization protocol described in Example 9. FIG. 7A: absolute number of cells isolated from the draining lymph nodes; FIG. 7B: percent OT-II transgenic CD4+ T-cells; and FIG. 6C: absolute number of OT-II transgenic CD4+ T-cells.

FIGS. 8A-F represent data derived from FACS analysis and cell counts of samples isolated from the draining lymph nodes taken at days 1, 5 or 9 post immunization of Flt3-ligand immunization protocols comparing various adjuvants and immune responses to vaccines (see Example 10). FIG. 8A: percent OT-I transgenic CD8+ T-cells at day 1 post immunization; FIG. 8B percent OT-I transgenic CD8+ T-cells at day 5 post immunization; FIG. 8C: percent OT-I transgenic CD8+ T-cells at day 9 post immunization; FIG. 8D: absolute number of OT-I transgenic CD8+ T-cells at day 1 post immunization; FIG. 8E absolute number of OT-I transgenic CD8+ T-cells at day 5 post immunization; and FIG. 8F: absolute number of OT-I transgenic CD8+ T-cells at day 9 post immunization.

FIGS. 9A-F represent data derived from FACS analysis and cell counts of samples isolated from the draining lymph nodes taken at days 1, 5 or 9 post immunization of Flt3-ligand immunization protocols comparing various adjuvants and immune responses to vaccines (see Example 10). FIG. 9A: absolute number of cells isolated from the draining lymph nodes at day 1 post immunization; FIG. 9B absolute number of cells isolated from the draining lymph nodes at day 5 post immunization; FIG. 9C: absolute number of cells isolated from the draining lymph nodes at day 9 post immunization; FIG. 9D: absolute number of OT-II transgenic CD4+ T-cells at day 1 post immunization; FIG. 9E absolute number of OT-II transgenic CD4+ T-cells at day 5 post immunization; and FIG. 9F: absolute number of.OT-II transgenic CD4+ T-cells at day 9 post immunization.

FIGS. 10A-F represent data derived from FACS analysis and cell counts of samples isolated from the draining lymph nodes taken at days 1, 5 or 9 post immunization of Flt3-ligand immunization protocols further comparing various adjuvants and immune responses to vaccines (see Example 10). FIG. 10A: percent OT-I transgenic CD8+ T-cells at day 1 post immunization; FIG. 10B percent OT-I transgenic CD8+ T-cells at day 5 post immunization; FIG. 10C: percent OT-I transgenic CD8+ T-cells at day 9 post immunization; FIG. 10D: absolute number of OT-I transgenic CD8+ T-cells at day 1 post immunization; FIG. 10E absolute number of OT-I transgenic CD8+ T-cells at day 5 post immunization; and FIG. 10F: absolute number of OT-I transgenic CD8+ T-cells at day 9 post immunization.

FIGS. 11A-F represent data derived from FACS analysis and cell counts of samples isolated from the draining lymph nodes taken at days 1, 5 or 9 post immunization of Flt3-ligand immunization protocols further comparing various adjuvants and immune responses to vaccines (see Example 10). FIG. 11A: absolute number of cells isolated from the draining lymph nodes at day 1 post immunization; FIG. 11B absolute number of cells isolated from the draining lymph nodes at day 5 post immunization; FIG. 11C: absolute number of cells isolated from the draining lymph nodes at day 9 post immunization; FIG. 11D: absolute number of OT-II transgenic CD4+ T-cells at day 1 post immunization; FIG. 11E absolute number of OT-II transgenic CD4+ T-cells at day 5 post immunization; and FIG. 11F: absolute number of OT-II transgenic CD4+ T-cells at day 9 post immunization.

FIG. 12 represents an “ex vivo” CTL assay of OT-I transgenic CD8+ T-cells isolated at day 5 post immunization from the Flt3-ligand immunization protocol described in Example 10. The transgenic CD8+ T-cells isolated from the draining lymph nodes were used directly in the CTL assay without in vitro expansion.

FIG. 13 represents an standard T-cell proliferation assay of OT-II transgenic CD4+ T-cells isolated at day 5 post immunization from the Flt3-ligand immunization protocol described in Example 10.

FIGS. 14A and 14B show that ten days pre-treatment with FL was effective at inducing expansion of OTI CD8+ T-cells post immunization, but 8, 6 and 4 days pre-treatment were also effective compared to the control groups.

FIGS. 15A and 15B illustrate that Flt3-L pretreatment followed by immunization with antigen in IFA adjuvant is effective for use with both peptide and protein antigens.

FIGS. 16A and 16B show that IL-15 given post immunization augmented OTI CD8+ CTL expansion and function when mice were pre-treated with Flt3-ligand and immunized with peptides in saline.

FIGS. 17A and 17B illustrate that treatment with IL-15 or anti-4-IBB immediately post immunization augments the size of the memory T cell pool. This effect was noted after immunization with Flt3-ligand plus peptides formulated in saline (gps 1-3) or Flt3-ligand plus peptides formulated in IFA (gps 7-9), but not after immunization in the absence of Flt3-ligand (gps 4-6).

### DETAILED DESCRIPTION OF THE INVENTION

Embodiments of the invention include methods of using Flt3-ligand in immunization protocols that exploit the unique attributes of Flt3-ligand. A Flt3-ligand immunization protocol, which is defined in more detail below, comprises, at least in part, administering Flt3-ligand to a subject and immunizing the subject with a vaccine. Embodiments of a Flt3-ligand immunization protocol have shown a surprising and unprecedented heightened and prolonged immune response to vaccination. This enhanced immune response is antigen-specific and is, at least in part, characterized by an increase in the number of antigen-specific effector cells, such as, but not limited to, CD8+ cytotoxic T-cells and CD4+ helper T-cells. Therefore, one embodiment of the invention provides for the use of an effective amount of Flt3-ligand to increase and/or mobilize antigen presenting cells in vivo and immunizing the subject with a vaccine, wherein the vaccine comprises an antigen and an adjuvant.

In other aspects, embodiments of the invention are directed to the in vivo use of Flt3-ligand to generate large numbers of intermediate cell types from hematopoietic progenitor cells and stem cells. Flt3-ligand (also referred to herein and in the art as Flt3-L or FL) is known to affect hematopoietic stem and progenitor cells. It has been shown that Flt3-ligand potently stimulates the generation of downstream or intermediate cells, such as myeloid precursor cells, monocytic cells, macrophages, B cells and dendritic cells from CD34⁺ bone marrow progenitors and stem cells. Large numbers of these intermediate cell types are not naturally found in vivo and can be generated by administering Flt3-ligand. Furthermore, Flt3-ligand has been shown to increase or mobilize dendritic cells in vivo, for example, in the subject's peripheral blood or other tissue or organs, such as the spleen, liver, lung and lymph nodes. By increasing the quantity of the subject's dendritic cells, such cells may themselves be used to present antigen to effector cells, such as T and B cells. Such enhancement in overall cell number can augment the immune response to antigen in the host. Flt3-ligand may be used, therefore, to boost the subject's lymphocyte-mediated (e.g., T cell and B cell mediated) or myeloid-mediated immune response to antigens thus enabling a more effective antigen-presentation to the subject's T cells. The overall response is a stronger and improved immune response and more effective immunization to the antigen.

Therefore, by modulating the quantity and character of the subject's antigen presenting cells (APCs), such as dendritic cells, macrophages, monocytes, B cells and the like, and exposing these APCs to antigens presented in the context of an adjuvant (i.e., a vaccine), a surprisingly robust antigen-specific immune response is generated.

Flt3-ligand immunization protocols refer to the administration of Flt3-ligand, a vaccine, optionally one or more auxiliary molecules, as well as other accompanying molecules and/or formulations (e.g., diluents, carriers, excipients and the like), to a subject for the prevention and/or treatment of a disease, disorder or infection. The Flt3-ligand, vaccine and other components described above may be administered in any dosage, order, frequency and temporal arrangement. One of skill in the art would recognize that varying these parameters to optimize treatment is routinely performed in the art. Therefore, for the purposes of this application, all such permutations and combinations of dosage, order, frequency and temporal arrangement are encompassed by the methods described herein. Further embodiments also include using Flt3-ligand in immunization regimens such as the “prime and boost” technique, wherein subjects are immunized with either a live virus vector expressing the antigen or a DNA-based vaccine (naked or plasmid) encoding the antigen and subsequently boosted with one or more antigens formulated in one or more adjuvants. Flt3-ligand may be administered prior to or at any time during or after the prime and boost regimen.

The studies presented herein demonstrate that a dramatic expansion of antigen-specific CD8+ T cells occurred when mice were treated with Flt3-ligand prior to immunization. For example, antigen-specific CD8+ T cells were approximately 34 times higher in mice receiving Flt3-ligand prior to immunization with antigen formulated in Incomplete Freund's Adjuvant (IFA) than mice not receiving Flt3-ligand, and 114-fold higher than mice only receiving antigen formulated in PBS. Five days after immunization, antigen-specific CTL had expanded to comprise 25-40% of all cells in the draining lymph nodes, equal to about 2.5-9×10⁶ cells. The further addition of auxiliary molecules, such as pGM-CSF and/or CD40-L, further augments and prolongs CD8+ T cell expansion.

The combination of Flt3-ligand pre-treatment and immunization with antigen formulated in adjuvants, such as those having a depot-like effect, had a dramatic effect on antigen-specific cytotoxic T-cell (CTL) generation. Notably, the CD8+ T cells expanded by the Flt3-ligand immunization protocol were functional, antigen-specific effector cells, as measured by standard CTL assays.

Remarkably, groups receiving Flt3-ligand prior to immunization, and optionally receiving an auxiliary molecule, had such a heightened immune response that CTL activity was measured in T-cell populations isolated directly from the draining lymph nodes, i.e., there was no requirement for in vitro restimulation to uncover antigen-specific CTL activity. These results demonstrate that the antigen-specific CD8+ CTL immune response generated in mice pre-treated with Flt3-ligand and subsequently vaccinated were as potent as those generated in an acute response to a viral infection.

Furthermore, subjects receiving Flt3-ligand prior to immunization had significantly higher levels of antigen-specific CD4+ T-helper cells that were biologically functional, as measured by standard proliferation and IFNγ detection assays.

In another series of studies, groups receiving Flt3-ligand pretreatment had consistently higher immune responses regardless of the adjuvant used in the vaccine formulation, i.e., Incomplete Freund's adjuvant (IFA), aluminum hydroxide (Alum) or Quil-A saponin (Quil-A). These findings demonstrate that Flt3-ligand immunization protocols enhance immune responses to antigens that are formulated in a variety of adjuvants and that Flt3-ligand immunization protocols enhance immune responses over immunizing with antigen/adjuvant alone. Furthermore, mice receiving Flt3-ligand pretreatment and vaccinated with antigen formulated in Alum or Quil-A maintained a higher percentage of CD4+ transgenic T-cells over time, which demonstrates that Flt3-ligand immunization protocols prolong a heightened immune response. Taken together, these results demonstrate that Flt3-ligand immunization protocols increase both the magnitude and duration of antigen-specific effector cell responses over standard vaccination techniques.

Additional studies show that pre-treatment with Flt3-ligand, i.e., administration of Flt3-ligand prior to administration of the vaccine, was effective over a range of immunization protocols. Notably, Flt3-L immunization protocols were effective in generating a CD8+ CTL effector cell reponse for both peptide and protein antigens. In other studies, auxilliary molecules, such as IL-15 given post immunization further augmented antigen specific CD8+ CTL expansion and function when mice were pre-treated with Flt3-ligand. It has been further shown that treatment with auxilliary molecules, such as IL-15 or anti-4-1BB immediately post immunization augments the size of the memory T-cell pool.

In alternative embodiments, Flt3-ligand immunization protocols and associated methods of treatment and/or prevention of disease and/or infection comprise vaccines having an adjuvant possessing depot-like and proinflammatory characteristics, such as emulsion or gel-based adjuvants, which includes, but is not limited to, Incomplete Freund's Adjuvant (IFA). One advantage of adjuvants having depot-like qualities is that they are highly effective with small antigens that otherwise would be rapidly cleared from the body. Without being bound by theory, adjuvants that tend to localize antigen in an initial depot and disseminate antigen to the draining lymph nodes may be effective because of the enhanced interaction of antigen and dendritic cells in the draining lymph nodes and subsequent activation of T-cells. Furthermore, the resident dendritic cells may be internalizing the antigen at or near the site of immunization and traveling to the draining lymph nodes and presenting the antigen to the T-cell rich areas, or dispersing antigen by exosomes to other dendritic cells in the lymph nodes, which in turn internalize the exosome and present the antigens to the T-cells.

Further advantages of treating and/or preventing diseases, disorders or infectious diseases using the methods described herein include, at least in part, enhanced immunogenicity of weakly immunogenic antigens, such as highly purified or recombinant antigens; potential reduction in the amount of antigen used; less frequent booster immunizations; improved efficacy; preferential stimulation of cell-mediated and/or humoral immunity; and, potential targeting of immune responses, such as targeting select cells of the Peyer's patches for mucosal immunity.

The term “subject” as used herein, refers to mammals. For example, mammals contemplated by the present invention include humans; primates; pets of all sorts, such as dogs, cats, etc.; domesticated animals, such as, sheep, cattle, goats, pigs, horses and the like; common laboratory animals, such as mice, rats, rabbits, guinea pigs, etc.; as well as captive animals, such as in a zoo or free wild animals. Throughout the specification, the term host is used interchangeably with subject.

Antigen presenting cells or APCs, as used herein are cells that display peptide fragments of protein antigens in association with MHC molecules on their cell surface. Some APCs may activate antigen-specific T cells. Examples of APCs include, but are not limited to dendritic cells, macrophages, monocytes, B cells and the like.

Effector cells, as used herein are cells that perform effector functions during an immune response, such as secreting cytokines and/or chemokines, killing microbes, recognizing and optionally killing infected or cancerous cells, as well as secreting antibodies. Examples include, but are not limited to T-cells (cytotoxic, helper, tumor-infiltrating), B-cells, NK cells, neutrophils, macrophages and dendritic cells.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

It is understood that this invention is not limited to the particular methodology, protocols, cell lines, animal species or genera, constructs, and reagents described, as such may vary. It is also understood that the terminology used herein is for the purpose of describing particular embodiments only, and is not intended to limit the scope of the present invention which will be limited only by the appended claims.

Flt3-Ligand

As used herein, the term Flt3-ligand refers to a genus of polypeptides that are described in U.S. Pat. No. 5,554,512 and U.S. Pat. No. 6,291,661, which are incorporated herein by reference. Forms of Flt3-ligand that may be used in the methods described herein include, but are not limited to, murine and human Flt3-ligand. A human Flt3-ligand cDNA was deposited with the American Type Culture Collection, Rockville, Md., USA (ATCC) on Aug. 6, 1993 and assigned accession number ATCC 69382 and a mouse Flt3-ligand cDNA was deposited on the same day and assigned accession number ATCC 69286. The deposits were made under the terms of the Budapest Treaty. Flt3-ligand is commercially available from Immunex Corporation, Seattle, Wash. Flt3-ligand can be made according to the methods described in the documents cited above. Flt3-ligand may be modified by the addition of one or more water-soluble polymers, such as, but not limited to, polyethylene glycol to increase bio-availability and/or pharmacokinetic half-life. In alternative embodiments, Flt3-binding proteins that mimic the biological effects of Flt3-ligand may be used in the immunization protocols described herein. For example, WO 95/27062 describes agonistic antibodies to Flt3, the receptor for Flt3-ligand, from which various Flt3 binding proteins can be prepared.

Particularly preferred forms of Flt3-ligand are biologically active, soluble forms of Flt3-ligand, and particularly those forms comprising the extracellular domain or one or more fragments of the extracellular domain. Soluble forms of Flt3-ligand are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble Flt3-ligand also includes those polypeptides which include part of the transmembrane region, provided that the soluble Flt3-ligand is capable of being secreted from a cell, and preferably retains the capacity to bind the Flt3 receptor and effectuate its biological effects. Soluble Flt3-ligand further includes oligomers or fusion polypeptides comprising the extracellular portion of at least one Flt3-ligand polypeptide, and fragments of any of these polypeptides that have Flt3-ligand polypeptide activity.

Human Flt3-ligand may comprise an amino acid sequence selected from the group consisting of amino acids 28 to Xaa of SEQ ID NO:1, wherein Xaa is an amino acid from 160 to 235. Alternative embodiments comprise an amino acid sequence selected from the group consisting of amino acids 27 to Xaa of SEQ ID NO:1, wherein Xaa is an amino acid from 160 to 235. Murine Flt3-ligand may comprise an amino acid sequence selected from the group consisting of amino acids 28 to Yaa of SEQ ID NO:2, wherein Yaa is an amino acid from 163 to 231. Embodiments of soluble human Flt3-ligand include: the amino acid sequence of residues 27-160 of SEQ ID NO:1 (inclusive), 28-160 of SEQ ID NO:1 (inclusive), 27-179 SEQ ID NO:1 (inclusive), 27-182 SEQ ID NO:1 (inclusive), 28-182 of SEQ ID NO:1 (inclusive), 27-235 SEQ ID NO:1 (inclusive) and 28-235 of SEQ ID NO:1 (inclusive). Embodiments of soluble murine Flt3-ligand include: the amino acid sequence of residues 28-163 of SEQ ID NO:2 (inclusive), the amino acid sequence of residues 28-188 of SEQ ID NO:2 (inclusive) and the amino acid sequence of residues 28-231 of SEQ ID NO:2 (inclusive).

Of course, Flt3-ligand variants that are substantially similar and retain comparable biological activity may be used in the methods described herein. The term “substantially similar” means a variant amino acid sequence preferably that is at least 80% identical to a native amino acid sequence, most preferably at least 90% identical. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website www.ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

Flt3-ligand variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring variants are also encompassed by the invention. Examples of such variants are proteins that result from alternate mRNA splicing events or from proteolytic cleavage of the native protein, wherein the native biological property is retained.

Further modifications in the Flt3-ligand peptide or Flt3-ligand DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the Flt3-ligand extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Preferably, such alteration, substitution, replacement, insertion or deletion does not diminish the biological activity of Flt3-ligand. One example is a variant that binds with essentially the same binding affinity as does the native form. Binding affinity can be measured by conventional procedures, e.g., as described in U.S. Pat. No. 5,512,457 and as set forth herein.

Additional Flt3-ligand derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E1 1 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Additional embodiments of Flt3-ligand that may be used in the methods described herein include oligomers or fusion polypeptides that contain a Flt3-ligand, one or more fragments of Flt3-ligand, or any of the derivative or variant forms of Flt3-ligand as disclosed herein, as well as in the U.S. patents listed above. In particular embodiments, the oligomers comprise soluble Flt3-ligand polypeptides. Oligomers can be in the form of covalently linked or non-covalently-linked multimers, including dimers, trimers, or higher oligomers. In an alternative embodiments, Flt3-ligand oligomers comprise multiple Flt3-ligand polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

Immunoglobulin-based Oligomers. Soluble Flt3-ligand and fragments thereof can be fused directly or through linker sequences to the Fc portion of an immunoglobulin. For a bivalent form of Flt3-ligand, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of Flt3-ligand is directed to a dimer comprising two fusion polypeptides created by fusing a Flt3-ligand to an Fc polypeptide derived from an antibody. A gene fusion encoding the Flt3-ligand/Fc fusion polypeptide is inserted into an appropriate expression vector. Flt3-ligand/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four Flt3-ligand extracellular regions.

Peptide-linker Based Oligomers. Alternatively, the oligomer is a fusion polypeptide comprising multiple Flt3-ligand polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble Flt3-ligand polypeptides, separated by peptide linkers. Suitable peptide linkers, their combination with other polypeptides, and their use are well known by those skilled in the art.

Leucine-Zippers. Another method for preparing the oligomers of Flt3-ligand involves use of a leucine zipper. Leucine zipper domains are peptides that promote oligomerization of the polypeptides in which they are found. Leucine zippers were originally identified in several DNA-binding polypeptides (Landschulz et al:, Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Flt3-ligand affects the growth of pluripotent haematopoietic stem and progenitor cells, as well as a number of lineages in the lymphoid and myeloid pathways. A synergistic effect with a wide range of colony stimulating factors, interleukins and soluble thrombopoietin, to promote growth and colony formation of committed and primitive progenitor cells has been demonstrated. In vivo administration of Flt3-ligand to mice results in a significant expansion of haematopoietic progenitor cells. In particular, Flt3-ligand causes a significant increase in the number of progenitors in the bone marrow (5-fold) and spleen (100-fold), as well as increasing the number of immature B cells in these tissues. A 200-500 fold increase in the number of haematopoietic progenitor cells has been reported in the peripheral blood following treatment. Flt3-ligand alone and in combination with other cytokines (IL-3, IL-6 or IL-7) has been shown to preferentially stimulate T cell development from the most primitive thymic progenitor cells. Additionally, in vitro studies have demonstrated that Flt3-ligand can induce the expansion of fetal liver, bone marrow or thymic natural killer (NK) cell progenitors, as well as costimulate (with IL-15 alone or a combination of IL-6/IL-7/IL-15) the generation of CD56+ NK cells from their progenitors. Flt3-ligand has also been shown to increase NK cell activity, NK cell proliferative responses, and generation of lymphocyte activated killer (LAK) cells, suggesting a potential role for Flt3-ligand in anti-cancer and anti-viral therapy. For a review of Flt3-ligand see “Flt3-ligand and Its Influence on Immune Reactivity” Cytokine, vol. 12, no. 2, pp 97-100 (2000).

Administration of Flt3-ligand (both in vivo and in vitro) causes targeted expansion of haematopoietic stem and progenitor cells resulting in a generalized expansion of dendritric cells (DC) in multiple tissue sites. Dendritic cells comprise a heterogeneous cell population with distinctive morphology and a widespread tissue distribution. The dendritic cell system and its role in immunity is reviewed by Steinman, R. M., Annu. Rev. Immunol., 9:271-296 (1991), and is incorporated herein by reference. Dendritic cells have a high capacity for sensitizing MHC-restricted T cells and are very effective at presenting antigens to T cells in situ, both self-antigens during T cell development and tolerance and foreign antigens during immunity.

As used herein, a dendritic cell, or DC, refers to any member of a diverse population of morphologically similar cell types found in lymphoid or non-lymphoid tissues. DCs are a class of “professional” antigen presenting cells, and have a high capacity for sensitizing MHC-restricted T cells. Depending upon their lineage and stage of maturation, DCs may be recognized by function, or by phenotype, particularly by cell surface phenotype. These cells are characterized by their distinctive morphology, phagocytic/endocytotic capacity, high levels of surface MHC-class II expression and ability to present antigen to T cells, particularly to naive T cells (Banchereau, et al., Annu. Rev. Immunol., 18:767-811, 2000 and U.S. Pat. No. 6,274,378, incorporated herein by reference for its description of such cells). For illustrative purposes only, DCs described herein may be characterized by veil-like projections and expression of the cell surface markers CD1a⁺, CD4⁺, CD86⁺, or HLA-DR⁺. Mature DCs are typically CD11c⁺, while precursors of DCs include those having the phenotype CD11c⁻, IL-3Rα^{low}; and those that are CD11c⁻ IL-3Rα^{high}. Treatment with GM-CSF in vivo preferentially expands CD11b^{high}, CD11c^{high} DC in mice, while Flt3-ligand has been shown to expand CD11c⁺ IL-3Rα^{low} DC, and CD11c³¹ IL-3Rα^{high} DC precursors in humans. Functionally, dendritic cells maybe identified by any convenient assay for determination of antigen presentation. Such assays may include testing the ability to stimulate antigen-primed or naive T cells by presentation of a test antigen, following by determination of T cell proliferation, release of IL-2, and the like.

Vaccines

A vaccine, as used herein, comprises one or more antigens formulated, combined, mixed, incorporated into and/or matrixed with one or more adjuvants, diluents, carriers and the like that is administered to a subject by any suitable route to induce protective and/or ameliorative immune responses to the antigen. A vaccine may be for the prevention of disease and administered prior to infection or onset of disease. Alternatively, the vaccine may be administered to the subject any number of times for therapeutic purposes after the subject has been diagnosed with a disease or infection. Antigens may be complexed with one or more haptens. A vaccine may comprise natural, derivatized, synthetic, recombinant or non-recombinant antigens. In addition, a vaccine includes live viral vectors containing polynucleotide sequences encoding one or more antigens. Examples of live viral vectors include retroviruses, adenoviruses, adeno-associated viruses, herpes viruses, alpha viruses, semliki forest viruses and sinbis viruses. In addition, a vaccine may comprises non-viral vectors, such as polynucleotide vaccines (commonly referred to as DNA vaccines) containing polynucleotide sequences encoding one or more antigens as well as necessary promoters, enhancers and other necessary regulatory elements. The polynucleotide vaccines may be naked polynucleotides and/or polynucleotides incorporated into liposomes and/or particle-mediated gene transfer. Other agents that may be used to facilitate delivery of the polynucleotide vaccine include polypeptides, peptides, polysaccharide conjugates, lipids, and the like. The polynucleotides may be DNA and/or RNA. Live viral vectors and polynucleotide vaccines may be formulated with a carrier or diluent, which are well known in the art.

The following are embodiments of adjuvants and antigens that may be used in any combination to form one or more vaccines, which in turn may be used in any or all embodiments of the Flt3-ligand immunization protocols and methods of treatment/prevention described herein.

A. Embodiments of Adjuvants Used in Flt3-Ligand Immunization Protocols.

The term adjuvant, for the purposes of this application, refers to any substance, that is distinct from the antigen which when incorporated into a vaccine acts generally to accelerate, prolong, enhance, augment and/or potentiate the host's immune response to the antigen. The host's immune response includes antigen-specific humoral and cell mediated immune responses. The host's immune response also includes immune responses that are not necessarily antigen-specific, but are involved in a protective and/or ameliorative immune response to the antigen, such as increased numbers and/or activation of NK cells, neutrophils, antigen presenting cells, and the like. Additional terms of art used to describe an adjuvant include immunomodulator, immunopotentiator and immunoenhancer. As used herein, an adjuvant includes all such compositions encompassed by the terms immunomodulator, immunopotentiator and immunoenhancer. It is understood that Flt3-ligand itself is considered an adjuvant because of its well-documented role in potentiating immune responses, and in particular potentiating a protective immune response against cancer. But, for the sake of clarity and to avoid confusion, Flt3-ligand will not be referred to herein as an adjuvant.

Adjuvants are thought to exert their biological effects by one or more mechanisms, including increasing the surface area of antigen; prolonging the retention of the antigen in the body thus allowing time for the lymphoid system to have access to the antigen; slowing the release of antigen; targeting antigen to macrophages; increasing antigen uptake; up-regulating antigen processing; stimulating cytokine release; stimulating B cell switching and maturation and/or eliminating immuno-suppressor cells; activating macrophages, dendritic cells, B cells and T cells; or otherwise eliciting non-specific activation of the cells of the immune system (see, for example, Warren et al., 1986, Annu Rev Immunol 4: 369). Generally speaking, adjuvants comprise a very heterogeneous group of compounds, but those of skill in the art have historically recognized a number of broad categories, such as oil emulsions (e.g., Freund's adjuvants), mineral compounds (e.g., alum), bacterial products (e.g., Bordetella pertussis), liposomes and immunostimulating complexes (ISCOMs).

Examples of adjuvants that may be used in making one or more vaccines that may be used in a Flt3-ligand immunization protocol as well as in methods of treatment and/or prevention include, but are not limited to: ADJUMER™ (polyphosphazene); aluminum phosphate gel; algal glucans; algammulin; aluminum hydroxide gel (alum); high protein adsorbency aluminum hydroxide gel; low viscosity aluminum hydroxide gel; AF or SPT (emulsion of squalane (5%), Tween 80(0.2%), Pluronic L121(1.25%), phosphate-buffered saline pH 7.4); AVRIDIE™ (propanediamine); BAY R1005™ ((N-(2-Deoxy-2-L-leucylamino-b-D-glucopyranosyl)-N-octadecyldodecanoylarmide hydroacetate); CALCITRIOL™ (1α, 25-dihydroxyvitamin D3); calcium phosphate gel; CAP™ (calcium phosphate nanoparticles); cholera holotoxin, cholera toxin A1-protein A-D fragment fusion protein, cholera toxin B subunit; CRL 1005 (Block Copolymer P1205); cytokine containing liposomes; DDA (dimethyldioctadecylammonium bromide); DHEA (dehydroepiandrosterone); DMPC (dimyristoyl phosphatidylcholine); DMPG (dimyristoyl phosphatidylglycerol); DOC/Alum Complex (deoxycholic Acid Sodium Salt); Freund's Complete Adjuvant; Freund's Incomplete Adjuvant; Gamma Inulin; Gerbu Adjuvant (mixture of: i) N-Acetylglucosaminyl-(P1-4)-N-acetylmuramyl-L-alanyl-D-glutamine (GMDP), ii) Dirnethyl dioctadecylammonium. chloride (DDA), iii) Zinc L-proline salt complex (ZnPro-8); GM-CSF; GMDP (N-acetylglucosaminyl-(b1-4)-N-acetylmuramyl-L-alanyl-D-isoglutamine); Imiquimod (1-(2-methypropyl)-IH-imidazo[4,5-c]quinolin-4-amine); ImmTher™ (N-acetylglucosaminyl-N-acetyhnuramyl-L-Ala-D-isoGlu-L-Ala-glycerol dipalmitate); DRVs (Immunoliposomes prepared from Dehydration-Rehyrdation Vesicles); Interferon-γ; Interleukin-1β; Interleukin-2; Interleukin-7; Interleukin-12; ISCOMS™ (Immune Stimulating Complexes); ISCOPREP 7.0.3. ™; Liposomes; LOXORIBINE™ (7-allyl-8-oxoguanosine); LT Oral Adjuvant™ (E. coli labile enterotoxin protoxin); Microspheres and Microparticles of any composition; MF59™; (squalene.water emulsion); MONTANIDE ISA 51™ (purified Incomplete Freund's Adjuvant); MONTANIDE ISA 720™ (metabolizable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxy-phosphoryloxy)) ethylamide, mono sodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glycerol dipalmitoyl); NAGO (Neuraminidase-galactose oxidase); Nanospheres or Nanoparticles of any composition; NISVs (Non-Ionic Surfactant Vesicles); PLEURAN™ (β-glucan); PLGA, PGA and PLA (homo-and co-polymers of lactic and glycolic acid; micro-/nanospheres); PLURONIC L121™; PMMA (polymethyl methacrylate); PODDS™ (oroteinoid microspheres); Polyethylene carbamate derivatives; Poly rA:Poly rU (Poly-adenylic acid-poly-uridylic acid complex); Polysorbate 80 (Tween 80); Protein Cochleates (Avanti Polar Lipids, Inc., Alabaster, AL); STIMULON™ (QS-21); Quil-A (Quil-A saponin); S-28463 (4-Amino-otec,-dimethyl-2-ethoxymethyl-lH-imidazo[4,5-c]quinoline-1-ethanol); SAF-1™ (Syntex Adjuvant Formulation); Sendai proteoliposomes and Sendai-containing lipid matrices; Span-85 (sorbitan trioleate); Specol (emulstion of Marcol 52, Span 85 and Tween 85); Squalene or Robane® (2,6,10,15,19,23-hexamethyltetracosane and 2,6,10,15,19, 23-hexamethyl-2,6,10,14,18,22 tetracosahexaene); Stearyl Tyrosine (Octadecyl tyrosine hydrochloride); Theramide® (N-acetylglucosaminyl-N-acetylinuramyl-L-Ala-D-isoGlu-L-Ala-dipalmitoxy propylamide); Theronyl-MDP (Termurtide™ or [thr ₁]-MDP; N-acetyl muramyl-L-threonyl-D-isoglutamine); Ty Particles (Ty-VLPs or virus like particles); Walter Reed Liposomes (Liposomes containing lipid A adsorbed to aluminum hydroxide), and the like.

In one embodiment, adjuvants having depot-like characteristics that disseminate antigen to the lymph nodes are used in a vaccine formulation, which are then used in Flt3-ligand immunization protocols and associated methods of treating and/or preventing disease and/or infection. In yet another embodiment, Incomplete Freund's Adjuvant is used in a vaccine formulation and used in a Flt3-ligand immunization protocol and associated methods of treating and/or preventing disease and/or infection.

In additional embodiments, molecules that activate the subject's T-helper cells to secrete IL-2, such as molecules having one or more MHC-II epitopes may be included in a vaccine formulation. In particular, T-helper activating molecules that enhance CD8+ CTL responses may be included in Flt3-ligand immunization protocols as an additional antigen in the vaccine formulation. For example, keyhole limpet hemocyanin (KLH) may be included in the vaccine formulation, or other suitable T-helper antigen known in the art, as well as whole cells, such as allogeneic cells.

B. Embodiments of Antigens Used in Flt3-Ligand Immunization Protocols

An antigen, as used herein comprises any molecule that may be bound by an antibody or T-cell receptor. If necessary, an antigen may be coupled to a hapten to make them immunogenic. Antigens, as used herein, encompasses immunogens, which are antigens that induce an immune response in a subject.

An antigen may be a product derived from allergens, viruses, bacteria, fungi, parasites and other infectious agents; molecules promoting autoimmune diseases, or tumor antigens, tumors and neoplastic organs and tissues are also included. More particularly, examples of antigens comprise whole inactivated organisms and cells, live attenuated organisms, whole cells (live or dead cells that may be autologous, allogeneic and/or syngeneic), cell fragments, subcellular fractions, cell membranes, and the like. It is understood that immunogenic portions or epitopes from the above-mentioned categories are included, which may be in the form of proteins, subunit proteins, multimeric subunit proteins, polypeptides, peptides, synthetic peptides, and the like, as well as all forms of carbohydrates and glycosylated proteins. Antigens may also be produced by recombinant DNA techniques that are well-known to those of ordinary skill in the art. Antigens specific to one or more types of cancer or infected cells, can be chosen from among those known in the art, as described below. Also, the antigen may be one that already exists within a subject, such as a tumor antigen, or a bacterial or viral antigen. The above-mentioned antigens and can be selected for their antigenicity or their immunogenicity, as determined by immunoassays or by their ability to generate an immune response. The term “immunogenicity” means relative effectiveness of an antigen to induce an immune response.

Representative of the antigens that can be used in various embodiments include, but are not limited to those described in Table 1 below. Potentially useful antigens, or derivatives thereof, can be identified by various criteria, such as the antigen's involvement in neutralization of a pathogen's infectivity (wherein it is desired to treat or prevent infection by such a pathogen) (Norrby, 1985, Summary, in Vaccines 85, Lemer, et al. (eds.), Cold Spring Harbor Laboratory, Cold Spring Harbor, N.Y., pp. 388-389), type or group specificity, recognition by subjects' antisera or immune cells, and/or the demonstration of protective effects of antisera or immune cells specific for the antigen.

In alternative embodiments, Flt3-ligand immunization protocols presented herein may be used in the treatment or prevention of cancer. Vaccines may comprise one or more tumor antigens formulated with an adjuvant, carrier and/or diluent. Tumor antigens (also referred to as cancer antigens) may be isolated, i.e., partially purified, cell-associated or some form of fusion protein. Cancer antigens may be derived from normal differentiation antigens; intronic sequences; alternative open reading frames; single-based mutations; and, proteins having aberrant post-transcriptional control of expression, chromosome rearrangement or processing. A large number of tumor antigens are well known in the art and may be used in Flt3-ligand immunization protocols, such as those described in Rosenberg, S. A., Nature, vol. 411, pp. 380-384, 17 May 2001 and Minev, B., et al., Pharmacol. Ther., Vol. 81, No. 2, pp. 121-139, 1999. In certain embodiments, cancer cells and pre-neoplastic cells used in vaccines are of mammalian origin, and in alternative embodiments, a human cancer cell can be used as a source of antigens. Cancer cells, as well as any antigen or subcellular fraction thereof, may be autologous or allogeneic. Cancer cells found in abnormally growing tissue, circulating leukemic cells, metastatic lesions as well as solid tumor tissue can be used. In addition, cell lines derived from a pre-neoplastic lesion, cancer tissues or cancer cells can also be used, provided that the cells of the cell line have at least one or more antigenic determinants in common with antigens on the target cancer cells.

Cancer and pre-neoplastic cells can be identified by any method known in the art. For example, cancer cells can be identified by morphology, enzyme assays, proliferation assays, cytogenetic characterization, DNA mapping, DNA sequencing, the presence of cancer-causing virus, or a history of exposure to mutagen or cancer-causing agent, imaging, etc. Cancer cells may also be obtained by surgery, endoscopy, or other biopsy techniques. Cancer cells can also be obtained or purified by any biochemical or immunological methods known in the art, such as but not limited to affinity chromatography and fluorescence activated cell sorting. Cancer tissues, cancer cells or cell lines may be obtained from a single individual or pooled from several individuals. It is not essential that clonal, homogeneous, or purified population of cancer cells be used. It is also not necessary to use cells of the ultimate target in vivo (e.g., cells from the tumor of the intended recipient), so long as at least one or more antigenic determinants on the target cancer cells is present on the cells used in the vaccine. In addition, cells derived from distant metastases may be used to prepare an immunogenic composition against the primary cancer. A mixture of cells can be used provided that a substantial number of cells in the mixture are cancer cells and share at least one antigenic determinant with the target cancer cell.

To determine immunogenicity or antigenicity of a putative antigen by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one aspect, antibody binding is detected by detecting a label on the primary antibody. In another aspect, the primary antibody is detected by detecting binding of a secondary antibody or reagent to the primary antibody. In a further aspect, the secondary antibody is labeled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

TABLE 1
Antigen

| Category | Some Specific Examples of Representative Antigens |
|---|---|
| Viruses | Rotavirus; foot and mouth disease; influenza, including influenza A and B; |
| | parainfluenza; Herpes species (Herpes simplex, Epstein-Barr virus, chicken pox, |
| | pseudorabies, cytomegalovirus); rabies; polio; hepatitis A; hepatitis B; hepatitis C; |
| | hepatitis E; measles; distemper; Venezuelan equine encephalomyelitis; feline leukemia |
| | virus; reovirus; respiratory syncytial virus; bovine respiratory syncytial virus; Lassa |
| | fever virus; polyoma tumor virus; parvovirus; canine parvovirus; papilloma virus; tick- |
| | borne encephalitis; rinderpest; human rhinovirus species; enterovirus species; Mengo |
| | virus; paramyxovirus; avian infectious bronchitis virus; HTLV 1; HIV-1; HIV-2; LCMV |
| | (lymphocytic choriomeningitis virus); adenovirus; togavirus (rubella, yellow fever, |
| | dengue fever); corona virus |
| Bacteria | Bordetella pertussis; Brucella abortis; Escherichia coli; Salmonella species including |
| | Salmonella typhi; streptococci; Vibrio species (V. cholera, V. parahaemolyticus); |
| | Shigella species; Pseudomonas species; Brucella species; Mycobacteria species |
| | (tuberculosis, avium, BCG, leprosy); pneumococci; staphlylococci; Enterobacter |
| | species; Rochalimaia henselae; Pasterurella species (P. haemolytica, P. multocida); |
| | Chlamydia species (C. trachomatis, C. psittaci, Lymphogranuloma venereum); Syphilis |
| | (Treponema pallidum); Haemophilus species; Mycoplasma species; Lyme disease |
| | (Borrelia burgdorferi); Legionnaires' disease; Botulism (Colstridium botulinum); |
| | Corynebacterium diphtheriae; Yersinia entercolitica |
| Ricketsial | Rocky mountain spotted fever; thyphus; Ehrlichia species |
| Infections | |
| Parasites | Malaria (Plasmodium falciparum, P. vivax, P. malariae); schistosomes; trypanosomes; |
| and | Leishmania species; filarial nematodes; trichomoniasis; sarcosporidiasis; Taenia species |
| Protozoa | (T. saginata, T. solium); Toxoplasma gondii; trichinelosis (Trichinella spiralis); |
| | coccidiosis (Eimeria species) |
| Fungi | Cryptococcus neoformans; Candida albicans; Apergillus fumigatus; |
| | coccidioidomycosis |
| Recombinant | Herpes simplex; Epstein-Barr virus; hepatitis B; pseudorabies; flavivirus (dengue, |
| Proteins | yellow fever);Neisseria gonorrhoeae; malaria: circumsporozoite protein, merozoite |
| | protein; trypanosome surface antigen protein; pertussis; alphaviruses; adenovirus |
| Proteins | Diphtheria toxoid; tetanus toxoid; meningococcal outer membrane protein (OMP); |
| | streptococcal M protein; hepatitis B; influenza hemagglutinin; cancer antigen; tumor |
| | antigens; toxins; exotoxins; neurotoxins; cytokines and cytokine receptors; monokines |
| | and monokine receptors |
| Synthetic | Malaria; influenza; foot and mouth disease virus; hepatitis B; hepatitis C |
| Peptides | |
| Poly- | Pneumococcal polysaccharide; Haemophilis influenza polyribosyl-ribitolphosphate |
| saccharides | (PRP); Neisseria meningitides; Pseudomonas aeruginosa; Klebsiella pneumoniae |
| Oligo- | Pneumococcal |
| saccharide | |
| Allergens | Plant pollens; animal dander; dust mites, Blatella species antigens (Bla g 1, 2, or 5), |
| | Periplaneta species antigens (Per a 1) |
| Human | Class I restricted antigens recognized by CD8 + lymphocytes: |
| Cancer Ags | Melanoma-Melanocyte Differentiation Antigens (MART-1/Melan A; gp 100/pmel- 17; |
| | Tyrosinase; Tyronsinase Related Protein-1; Tyronsinase Related Protein-2; Melanocyte- |
| | Stimulating Hormone Receptor); Cancer-Testes Antigens (MAGE-1; MAGE-2; MAGE- |
| | 3; MAGE-12, BAGE; CAGE, NYESO-1); Mutated Antigens (β-catenin; MUM-1; |
| | CDK-4; Caspase-8; KIA 0205; HLA-A2-R1701); and Non-Mutated Shared Antigens |
| | Overexpressed on Cancers (α-Fetoprotein; Telomerase Catalytic Protein; G-250; MUC- |
| | 1; Carcinoembryonic antigen; p53; Her-2/neu). |
| | Class II restricted antigens recognized by CD4 + lymphocytes: |
| | Epitopes from Non-Mutated Proteins (gp 100; MAGE-1; MAGE-3; Tyrosinase; NY- |
| | ESO-1) and Epitopes from Mutated Proteins (Triosephosphate isomerase; CDC-27; |
| | LDLR-FUT) |
| Infectious | Bacteria: Helicobacter pylori (gastric cancer and lymphoma) |
| agents as | Viruses: Human Papliloma Virus (cervical and anal cancers); Hepatitis B and C Virus |
| cancer Ags | (liver cancer); HIV (Kaposi's sarcoma, non-Hodgkin's lymphoma); Human-Herpesvirus |
| | Type B (Kaposi's sarcoma); Epstein-Barr Virus (lymphomas); Human T-Cell |
| | Lymphotropic Viirus (adult T-cell leukemia) |
| | Parasite: Schistosomes (bladder cancer); Liver Flukes (cholangiocarcinoma) |

Vaccines, as used herein, also include antigens formulated with diluents, excipients and/or carriers in addition to, or rather than, an adjuvant. Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The antigen can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like.

Auxiliary Molecules

An auxiliary molecule, as defined herein, is a molecule that is optionally administered to a subject in a Flt3-ligand immunization protocol, which may be used in the treatment of cancer, infectious disease and symptoms thereof. An auxiliary molecule may act to accelerate, prolong, enhance, augment or potentiate the host's immune response to an antigen by any mechanism. For example, cytokines, growth factors and the like will be useful in further enhancing or modulating an immune response. Cytokines include, but are not limited to those selected from the group comprising Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

Typically, cytokines, chemokines and cell surface molecules exert their biological effects by binding their cognate on the surface of a cell and transducing an intracellular signal. Thus, binding proteins can be used in the Flt3-ligand immunization protocols. Binding proteins are agonistic molecules that mimic the biological effects of auxiliary molecules. For example, binding proteins, such as antibodies, bind appropriate receptors and transduce a signal equal or similar to the auxiliary molecule. Furthermore, auxiliary molecules used in Flt3-ligand immunization protocols include analogs of the above-mentioned molecules that have an amino acid sequence that is substantially similar to the native amino acid sequences and which are biologically active in that they are capable of binding their cognate and transducing a biological signal. Such analogs can be prepared and tested by methods that are known in the art and as described herein.

Therapeutic Applications

Flt3-ligand immunization protocols provide compositions and methods for preventing or treating a disease, disorder and/or infection in a subject. The terms “treat,” “treating” and “treatment” used herein includes curative, preventative (e.g., prophylactic), palliative and/or ameliorative treatment. As previously mentioned, Flt3-ligand and auxiliary molecules may be in any form described herein, such as native polypeptides, variants, derivatives, oligomers, and biologically active fragments. In particular embodiments, Flt3-ligand and auxiliary molecules comprise a soluble polypeptide or a soluble, oligomeric form.

One of skill in the art recognizes that it is a fundamental tenet of immunology and vaccinology that an immunoprotective response is typically generated against a disease by immunizing the subject with an antigen that is immunologically relevant to the disease being treated. Therefore, it is understood that the methods of treating cancer, infectious disease and the like include vaccines having antigens immunologically relevant to the disease being prevented or treated. For example, cancer vaccines would comprise one or more cancer antigens and an adjuvant, and more particularly, prostate cancer vaccines would comprise one or more prostate cancer antigens and an adjuvant, and so on.

For in vivo administration to subjects and especially humans, Flt3-ligand can be formulated according to known methods used to prepare pharmaceutical compositions. Flt3-ligand can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. The term pharmaceutically acceptable means a non-toxic material that does not interfere with the effectiveness of the biological activity of the active ingredient(s). Suitable carriers and their formulations are described in Remington's Pharmaceutical Sciences, 16th ed. 1980, Mack Publishing Co. In addition, such compositions can contain Flt3-ligand complexed with polyethylene glycol (PEG)—or other such compounds to increase solubility and/or pharmacokinetic half-life, metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of Flt3-ligand.

Flt3-ligand pharmaceutical compositions can be administered topically, parenterally, or by inhalation. The term “parenteral” includes subcutaneous injections, intravenous, intramuscular, intracisternal injection, or infusion techniques. These compositions will typically contain an effective amount of the Flt3-ligand, alone or in combination with an effective amount of any other active material. Such dosages and desired drug concentrations contained in the compositions may vary depending upon many factors, including the intended use, subject's body weight and age, and route of administration. Preliminary doses can be determined according to animal tests, and the scaling of dosages for human administration can be performed according to art-accepted practices. Keeping the above description in mind, typical dosages of Flt3-ligand may range from about 10 μg per square meter to about 1000 μg per square meter. A preferred dose range is on the order of about 100 μg per square meter to about 300 μg per square meter.

In practicing Flt3-ligand immunization protocols and methods of treatment and/or prevention, a therapeutically effective amount of Flt3-ligand, a vaccine, and optionally an auxiliary molecule are administered to a subject. As used herein, the term “effective amount” means the total amount of each therapeutic agent (i.e., Flt3-ligand, a vaccine, and optionally an auxiliary molecule) or other active component that is sufficient to show a meaningful benefit to the subject, i.e., enhanced immune response, treatment, healing, prevention or amelioration of the relevant medical condition (disease, infection, etc.), or an increase in rate of treatment, healing, prevention or amelioration of such conditions. When “effective amount” is applied to an individual therapeutic agent administered alone, the term refers to that therapeutic agent alone. When applied to a combination, the term refers to combined amounts of the ingredients that result in the therapeutic effect, whether administered in combination, serially or simultaneously. As used herein, the phrase “administering an effective amount” of a therapeutic agent means that the subject is treated with said therapeutic agent(s) in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent(s). Preferably, the baseline examination is done within about 60 days of administering the first dose. If the therapeutic agent(s) is/are being administered to treat acute symptoms, the first dose is administered as soon as practically possible. Improvement is induced by administering therapeutic agents until the subject manifests an improvement over baseline for the chosen indicator or indicators. In treating chronic conditions, this degree of improvement is obtained by repeatedly administering the therapeutic agents over a period of at least a month or more, e.g., for one, two, or three months or longer, or indefinitely. A period of one to six weeks, or even a single dose, may be sufficient for treating certain conditions. One of skill in the art would particularize the treatment to suit the subjects needs. Although the extent of the subject's illness after treatment may appear improved according to one or more indicators, treatment may be continued indefinitely at the same level or at a reduced dose or frequency. Once treatment has been reduced or discontinued, it later may be resumed at the original level if symptoms should reappear.

One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the therapeutic agents used to practice the methods described herein should contain about 0.01 ng to about 100 mg (alternative embodiments have about 0.1 ng to about 10 mg, and other embodiments have about 0.1 microgram to about 1 mg) of polypeptide of the present invention per kg body weight. If a route of administration other than injection is used, the dose is appropriately adjusted in accord with standard medical practices. For incurable chronic conditions, the regimen can be continued indefinitely, with adjustments being made to dose and frequency if such are deemed necessary by the patient's physician.

Pharmaceutical compositions may also comprise Flt3-ligand combined with one or more auxiliary molecules, as well as a pharmaceutically acceptable diluent, carrier, or excipient, are encompassed by the invention. Alternatively, the auxiliary molecules may be formulated as a separate pharmaceutical composition. Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. Flt3-ligand and/or auxiliary molecules can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323. Such compositions will influence the physical state, solubility, stability, rate of in vivo release, and rate of in vivo clearance, and are thus chosen according to the intended application, so that the characteristics of the carrier will depend on the selected route of administration.

In one embodiment, sustained-release forms of Flt3-ligand and auxiliary molecules are used. Sustained-release forms suitable for use in the disclosed methods include, but are not limited to, Flt3-ligand and auxiliary molecules that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

One type of sustained release technology that may be used in administering soluble Flt3-L therapeutic compositions is that utilizing hydrogel materials, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59, 1994) and to prevent thrombosis and vessel narrowing following vascular injury (Hill-West et al., Proc. Natl. Acad. Sci. USA 91:5967, 1994). Polypeptides can be incorporated into such hydrogels to provide sustained, localized release of active agents (West and Hubbel, Reactive Polymers 25:139, 1995; Hill-West et al., J. Surg. Res. 58:759; 1995). The sustained, localized release Flt3-L when incorporated into hydrogels would be amplified by the long half life of Flt3-L.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms; e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. J. Pharmaceutics 63: 135-44 (leuprolide acetate); Braquet et al. (1989), J. Cardiovasc. Pharmacol. 13 (suppl. 5): s. 143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

Alternative embodiments further include administration of Flt3-ligand concurrently with one or more other auxiliary molecules administered to the same subject. It is understood that the Flt3-ligand and auxiliary molecule(s) are administered as pharmaceutical compositions. Concurrent administration encompasses simultaneous or sequential treatment with Flt3-ligand and/or auxiliary molecules, as well as protocols in which the components are alternated, or wherein one component is administered long-term and the other(s) are administered intermittently. Components, i.e., Flt3-ligand and one or more auxiliary molecules, can be administered in the same or in separate compositions, and by the same or different routes of administration. Examples of auxiliary molecules that can be administered concurrently with Flt3-ligand include: cytokines, growth factors and the like will be useful in further enhancing or modulating an immune response. Cytokines include, but are not limited to those selected from the group comprising Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

Routes of Administration. Any efficacious route of administration can be used to therapeutically administer Flt3-ligand, one or more auxiliary molecules and one or more vaccines. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Cells may also be cultured ex vivo in the presence of Flt3-ligand, one or more auxiliary molecules and one or more vaccines in order to modulate cell proliferation or to produce a desired effect on or activity in such cells. Treated cells can then be introduced in vivo for therapeutic purposes. When Flt3-ligand, one or more auxiliary molecules and one or more vaccines are administered to a subject, these can be administered by the same or by different routes, and can be administered simultaneously, separately or sequentially.

Oral Administration. When a therapeutically effective amount of Flt3-ligand, one or more auxiliary molecules and one or more vaccines are administered orally, they may be in the form of a tablet, capsule, powder, solution or elixir. When administered in tablet form, the Flt3-ligand, one or more auxiliary molecules and one or more vaccines can additionally contain a solid carrier such as a gelatin or an adjuvant. The tablet, capsule, and powder contain from about 5 to 95% polypeptide of the present invention, and preferably from about 25 to 90% polypeptide of the present invention. Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference in its entirety. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference in its entirety. In general, the formulation will include Flt3-L and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., , pp. 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties. For oral delivery dosage forms, it is also possible to use a salt of a modified aliphatic amino acid, such as sodium N-(8-[2-hydroxybenzoyl] amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

When administered in liquid form, a liquid carrier such as water, petroleum, oils of animal or plant origin such as peanut oil, mineral oil, soybean oil, or sesame oil, or synthetic oils can be added. The liquid form of Flt3-ligand, one or more auxiliary molecules and one or more vaccines can further contain physiological saline solution, dextrose or other saccharide solution, or glycols such as ethylene glycol, propylene glycol or polyethylene glycol.

Intravenous Administration. When a therapeutically effective amount of Flt3-ligand, one or more auxiliary molecules and one or more vaccines is administered by intravenous, cutaneous or subcutaneous injection, Flt3-ligand, one or more auxiliary molecules and one or more vaccines may be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present invention can also contain stabilizers, preservatives, buffers, antioxidants, or other additives known to those of skill in the art. The duration of intravenous therapy using the pharmaceutical compositions of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical compositions of the present invention.

Tissue Administration. Flt3-ligand, one or more auxiliary molecules and one or more vaccines of the present invention may be administered topically, systematically, or locally as an implant or device. When administered, the Flt3-ligand, one or more auxiliary molecules and one or more vaccines is, of course, in a pyrogen-free, physiologically acceptable form. Further, the Flt3-ligand, one or more auxiliary molecules and one or more vaccines can be encapsulated or injected in a viscous form for delivery to a desired site. Topical administration of Flt3-ligand, one or more auxiliary molecules and/or one or more vaccines is also envisioned for alternative embodiments of Flt3-ligand immunization protocols.

Flt3-ligand immunization protocols may be used in the treatment and/or prevention of viral infection, including infection by: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunga viruses, phleboviruses and Nairo viruses); Arena viridae (hemorrhagic fever viruses); Reoviridae (e.g., reoviruses, orbiviuises and rotaviruses); Birnaviridae; Hepadnaviridae (Hepatitis B virus); Parvoviridae (parvovirusies); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Flt3-ligand mmunization protocols may be used in the treatment and/or prevention of infection by bacterium, including infection by: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

In alternative embodiments, Flt3-ligand immunization protocols may be used to treat or immunize subjects against infectious unicellular organisms, including infection by: schistosomes; trypanosomes; Leishmania species; filarial nematodes; trichomoniasis; sarcosporidiasis; Taenia saginata, Taenia solium, Cryptococcus neoformans, Apergillus fumigatus, Histoplasma capsulatum, Coccidiodes immitis, trichinelosis, Blastomyces dermatitidis, Chlamydia trachomatis, Candida albicans, Plasmodium falciparum, Plasmodium vivax, Plasmodium malariae, and Toxoplasma gondii and the like.

In additional embodiments, Flt3-ligand immunization protocols may be used to treat or vaccinate subjects against cancer, such as, but not limited to: mammalian sarcomas and carcinomas, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, serminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, such as acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. Various lymphoproliferative disorders also are treatable including autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

Flt3-ligand immunization protocols may also be used in combination with other recognized treatments known in the art. For example, in the treatment of cancer, Flt3-ligand immunization protocols may be used in combination with surgery, chemotherapy, radiation therapy, adoptive immunotherapy and the like. One underlying rationale is that tumor bulk is minimal and/or tumor cells are shed into the circulation during and following surgery and immunotherapy through Flt3-ligand immunization protocols may be more effective in this situation. In a specific embodiment, the preventive and therapeutic utility of the invention is directed at enhancing the immunocompetence of the cancer subject either before surgery, at or after surgery, and at inducing tumor-specific immunity to cancer cells, with the objective being inhibition of cancer, and with the ultimate clinical objective being cancer regression and/or eradication.

The effect of Flt3-ligand immunization protocols on progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor specific antigens; d) changes in the morphology of tumors using techniques such as a computed tomographic (CT) scan; e) changes in levels of putative biomarkers of risk for a particular cancer in individuals at high risk, and f) changes in the morphology of tumors. Alternatively, immune responses to the antigen of interest may be measured using standard techniques, such as CTL assays, proliferation assays, antibody capture assays, and the like.

In alternative embodiments, Flt3-ligand immunization protocols may be combined with adoptive immunotherapy using antigen presenting cells (APC) sensitized with one or more of the antigens described above. Adoptive immunotherapy refers to a therapeutic approach for treating infectious diseases or cancer in which immune cells are administered to a host with the aim that the cells mediate specific immunity, either directly or indirectly, to the infected cells or tumor cells and/or antigenic components, and result in treatment of the infectious disease or regression of the tumor. In one embodiment, the antigen-sensitized APC can be administered prior to, concurrently with or after administration of a vaccine. Furthermore, the mode of administration for adoptive immunotherapy can be varied, including but not limited to, e.g., subcutaneously, intravenously,intraperitoneally, intramuscularly, intradermally or mucosally.

The following is one embodiment of a Flt3-ligand immunization protocol, which relates to a method of immunizing a subject, comprising the steps of:

(a) administering Flt3-ligand to a subject;

(b) optionally administering an auxiliary molecule; and,

(c) administering a vaccine to the subject, wherein the vaccine comprises an antigen and an adjuvant, wherein, Flt3-ligand is administered prior to, concurrent with and/or subsequent to administration of the vaccine, and wherein the auxiliary molecule is administered prior to, concurrent with and/or subsequent to administration of the vaccine, and wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

The following is one embodiment of a Flt3-ligand immunization protocol, which relates to a method of treating and/or preventing cancer, viral infection, bacterial infection or infection by a unicellular organism in a subject suffering from cancer, viral infection, bacterial infection or infection by a unicellular organism, comprising the steps of: (a) administering Flt3-ligand to the subject, (b) optionally administering an auxiliary molecules to the subject, and (c) administering a vaccine to the subject.

The following is one embodiment of a Flt3-ligand immunization protocol, which relates to a method of treating cancer in a subject having cancer, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more cancer antigens formulated in an adjuvant.

The following is one embodiment of a Flt3-ligand immunization protocol, which relates to a method of treating viral infection in a subject having a viral infection, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more viral antigens formulated in an adjuvant.

The following is one embodiment of a Flt3-ligand immunization protocol, which relates to a method of treating bacterial infection in a subject having a bacterial infection, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more bacterial antigens formulated in an adjuvant.

The following is one embodiment of a Flt3-ligand immunization protocol, which relates to a method of treating infection by an unicellular organism in a subject having an infection by an unicellular organism, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more antigens from an unicellular organism formulated in an adjuvant.

The following is one embodiment of a Flt3-ligand immunization protocol, which relates to a method of enhancing an antigen-specific immune response to a cancer antigen in a subject suffering from cancer, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more cancer antigens formulated in an adjuvant.

The following is one embodiment of a Flt3-ligand immunization protocol, which relates to a method of enhancing an antigen-specific immune response to a viral antigen in a subject suffering from a viral infection, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more viral antigens formulated in an adjuvant.

The following is one embodiment of a Flt3-ligand immunization protocol, which relates to a method of enhancing an antigen-specific immune response to a bacterial antigen in a subject having a bacterial infection, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more bacterial antigens formulated in an adjuvant.

The following is one embodiment of a Flt3-ligand immunization protocol, which relates to a method of enhancing an antigen-specific immune response to an unicellular organism in a subject having an infection by an unicellular organism, comprising the steps of: (a) administering Flt3-ligand to the subject, (b) optionally administering a pharmaceutical composition comprising one or more auxiliary molecules to the subject, and (c) administering a vaccine to the subject, wherein the vaccine comprises one or more antigens from an unicellular organism formulated in an adjuvant.

The following is one embodiment of a Flt3-ligand immunization protocol, which relates to a method of enhancing an immune response to an antigen in a subject, comprising the steps of:

(a) administering Flt3-ligand to a subject;

(b) optionally administering an auxiliary molecule; and,

(c) administering a vaccine to the subject, wherein the vaccine comprises an antigen and an adjuvant.

The following is one embodiment of a Flt3-ligand immunization protocol, which relates to a method of enhancing an antigen-specific cytotoxic T-cell immune response to an antigen in a subject, comprising the steps of:

(a) administering Flt3-ligand to a subject;

(b) optionally administering an auxiliary molecule; and,

(c) administering a vaccine to the subject, wherein the vaccine comprises an antigen and an adjuvant.

The following is one embodiment of a Flt3-ligand immunization protocol, which relates to a method of enhancing an antigen-specific T-helper immune response to an antigen in a subject, comprising the steps of:

(a) administering Flt3-ligand to a subject;

(b) optionally administering an auxiliary molecule; and,

(c) administering a vaccine to the subject, wherein the vaccine comprises an antigen and an adjuvant.

In each of the foregoing embodiments, FIt3-ligand may be administered prior to, concurrent with and/or subsequent to administration of the vaccine and the auxiliary molecule may be administered prior to, concurrent with and/or subsequent to administration of the vaccine. In addition, in each of the foregoing embodiments, the auxiliary molecule may be selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

In Vivo Evaluation of Antigens and/or Vaccines in Animal Models and Laboratory Animals

All the foregoing methods of using Flt3-ligand immunization protocols are amenable to in vivo evaluation of antigens and/or vaccines in animal models and laboratory animals. For example, a growing list of tumor-associated antigens (TAAs) and tumor-specific antigens (TSAs) require evaluation in animal studies prior to Phase 1 testing in humans. It is understood that antigens other than those associated with cancer, such as those listed above, may be used in in vivo evaluation of antigens and/or vaccines.

Therefore, methods of evaluating immune responses to an antigen in animal models and/or laboratory animals (i.e., subjects) are provided. The following is one embodiment of a Flt3-ligand immunization protocol, which relates to a method of evaluating the immune responses to an antigen in a subject, comprising the steps of:

(a) administering Flt3-ligand to a subject;

(b) optionally administering an auxiliary molecule;

(c) administering an antigen to the subject, wherein the antigen may optionally be formulated with an adjuvant; and,

(d) evaluating the subject's immune responses to the antigen.

As with other embodiments, Flt3-ligand and the optional auxiliary molecules may be administered prior to, concurrent with and/or subsequent to administration of the vaccine. The auxiliary molecule may be selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

Evaluating the subject's immune responses can be monitored by any methods known in the art, including but not limited to, measuring: a) delayed hypersensitivity as an assessment of cellular immunity; b) activity of cytolytic T-lymphocytes in vitro; c) proliferative activity of T-helper lymphocytes in vitro; d) levels of antigen-specific antibodies, as well as isotypes of antigen specific antibodies; e) changes in the morphology of tissues, such as tumors; e) changes in levels of a surrogate marker for a particular disease or infection; and, (f) antigen-induced cytokine and/or chemokine production.

Flt3-Ligand in the Treatment of Allergies

FIt3-L may be used in the treatment of allergies. The Flt3-L immunization protocols described throughout this specification have direct utility in the allergen-specific immunotherapy of allergies. Allergen-specific immunotherapy is defined as the administration of increasing doses of an allergen vaccine to a subject having one or more allergies in order to reach a dose effective to improve symptoms associated with subsequent exposure to the causative allergen. Immunotherapy of allergies includes FIt3-L immunization protocols and allergen-specific immunotherapy of allergies and/or desensitation therapy that have been modified to include administration of F13-L.

Any suitable in vivo and in vitro techniques employed in the art of allergological diagnosis may be used to diagnose a subject, including more conventional tests such as but not limited to intradermal Serial Endpoint Testing (SET), radioallergosorbent assay (RAST), RAST Spot Test, Histamine Radioensymatic Assays, in vitro IgE and IgG assays, spontaneous synthesis assays, as well as other assays known in the art.

Allergy vaccines are well-known in the art, and can generally be defined as comprising at least one allergen and any suitable carrier, diluent, exipient, stabilizer and optional adjuvant. An allergen is defined herein as any art-recognized allergen; modified allergens (modified by such methods as, but not limited to, urea, PEG/PVA, deglycosylation, polysaccharides and/or photooxidation); allergoids (modified by such methods as, but not limited to, glutaraldehyde and/or formaldehyde treatment with or without tyrosine absorbtion); monovalent allogenic extracts; allergen polymers; conjugated allergens; allergen-muramylpepti des of allergens; allergen mycoloilmuramylpeptide conjugates; allergen-pullulan compounds; conjugates of allergen and hapten(s); conjugates of allergen, hapten(s) and hydrophilic polymers; urea denaturod antigens; recombinant allergens; mutagenized recombinant allergens; genetically-engineered allergens such as hypoallergens and/or hypoallergenic derivatives (e.g., molecules with reduced IgE-binding epitopes but preserving T-cell epitopes and epitopes for the induction of IgG antibodies that may serve as blocking antibodies);

The allergy vaccine and Flt3-L may be adminstered in any efficacious manner and route described herein. Dosing and administration of the allergen vaccine and Flt3-L may be determined by qualified physicians.

Flt3-L immunization protocols can be used in allergy immunotherapy for any treatable allergies, which includes, but is not limited to: insect allergies and insect bites and/or stings (dust mites, ants, spiders, flies, bees, wasps, mosquitoes, gnats and the like); animal allergies (fur, dander, excrement, etc. of domesticated and wild, such as, but not limited to: dogs, cats, birds, rodents, cows, sheep, horse, pigs, goats and the like); allergic bronchitis, balsam allergy; candida allergy; carpet and/or fabric allergy; food allergies or sensitivities; allergies to metals; colophony allergy; disinfectant allergy; fertilizer allergy; formaldehyde allergy; gas allergy; glue allergy; allogeneic serum allergy; jewelry allergy; mercapto allergy; mold and/or mildew allergy; paint allergy; paper allergy; parabens allergy; perfume allergy; pesticide allergy; plastic allergy; shampoo allergy; soap allergy; thiuram allergy; tobacco allergy; wheat allergy; yeast allergy; allergic dermatitis; allergic rhinitis; aspirin sensitivity; asthma; atopic dermatitis; contact dermatitis; cosmetic allergy; cows milk allergy; dermatitis; dust allergy; pollen allergy; eczema; grass allergy; salicylate sensitivity and the like.

As described in detail above, Fit3-L may be administered to a subject prior to, concurrent with and/or subsequent to the administration of a allergy vaccine and optional auxiliary molecules. In one embodiment, Flt3-L is administered to the subject once a day, everyday or every 2^{nd}, 3^{rd}, 4^{th}, 5^{th}, 6^{th} or 7^{th} day for 1, 2, 3, 4, 5, 6, 7, 8, 9, 10, 11, 12, 13, 14, 15, 16, 17, 18, 19 or 20 consecutive days prior to, concurrent with and/or subsequent to vaccination. Of course, all the embodiments of Flt3-L immunization protocols desribed above may be adapted to the treatment of allergies. Furthermore, existing allergy immunization regimens known in the art may be modified to include administration of Flt3-L.

In one embodiment, Flt3-ligand immunization protocols relate to a method of treating allgeries in a subject having one or more allergies, comprising the steps of:

(a) administering Flt3-ligand to the subject;

(b) optionally administering an auxiliary molecule; and,

(c) administering an allergy vaccine to the subject, wherein Flt3-ligand is administered prior to, concurrent with and/or subsequent to administration of the vaccine, and wherein the auxiliary molecule is administered prior to, concurrent with and/or subsequent to administration of the vaccine, and wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

As described above, Flt3-L expands hematopoietic stem and progenitor cells as well as various types of immune cells, especially dendritic cells. Furthermore, Flt3-L has the capacity to expand Th1-type dendritic cells. Therefore, Flt3-L immunization protocols in allergy immunotherapy can drive the subject's immune response from a typical Th2 respone to a Th1-type response. This shift in cytokine profile and immune response will drive IgG production over IgE production, decrease circulating levels of IL-4, decrease recruitment and activation of eosinophils, as well as decrease proliferation of mast cells. As a result, subsequent allergen exposure does not provoke an allergic reaction.

The efficacy of allergen-specific immunotherapy using a Flt3-L immunization protocol may be evaluated by standard methods and techniques known in the art, such as but not limited to measurement of allergen-specific IgG and IgE antibodies from the patient.

Patients undergoing allergen-specific immunotherapy in a Flt3-L immuniztion protocol may also be treated in combination with one or more conventional therapies, such as but no limited to, antihistamines, decongestants, steroids, analgesics, cough suppressants, and the like.

The relevant disclosures of all publications cited herein are specifically incorporated by reference. The following examples are provided to illustrate particular embodiments and not to limit the scope of the invention.

### EXAMPLES

### Example 1

This Example describes a method for using Flt3-ligand for dendritic cell expansion. Prior to cell collection, it may be desirable to mobilize or increase the numbers of circulating PBPC and PBSC. Mobilization can improve PBPC and PBSC collection, and is achievable through the intravenous administration of Flt3-ligand or sargramostim (Leukine®, Immunex Corporation, Seattle, Wash.) to the patients prior to collection of such cells. Other growth factors such as CSF-1, GM-CSF, c-kit ligand, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF, FGF and combinations thereof, can be likewise administered in sequence, or in concurrent combination with Flt3-ligand. Mobilized or non-mobilized PBPC and PBSC are collected using apheresis procedures known in the art. See, for example, Bishop et al., Blood, vol. 83, No. 2, pp. 610-616 (1994). Briefly, PBPC and PBSC are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg patient are collected. Aliquots of collected PBPC and PBSC are assayed for granulocyte-macrophage colony-forming unit (CFU-GM) content by diluting approximately 1:6 with Hank's balanced salt solution without calcium or magnesium (HBSS) and layering over lymphocyte separation medium (Organon Teknika, Durham, N.C.). Following centrifugation, MNC at the interface are collected, washed and resuspended in HBSS. One milliliter aliquots containing approximately 300,000 MNC, modified McCoy's 5A medium, 0.3% agar, 200 U/mL recombinant human GM-CSF, 200 u/mL recombinant human IL-3, and 200 u/mL recombinant human G-CSF are cultured at 37° C. in 5% CO₂ in fully humidified air for 14 days. Optionally, Flt3-ligand or GM-CSF/IL-3 fusion molecules (PIXY 321) may be added to the cultures. These cultures are stained with Wright's stain, and CFU-GM colonies are scored using a dissecting microscope (Ward et al., Exp. Hematol., 16:358 (1988). Alternatively, CFU-GM colonies can be assayed using the CD34/CD33 flow cytometry method of Siena et al., Blood, Vol. 77, No. 2, pp 400-409 (1991), or any other method known in the art.

CFU-GM containing cultures are frozen in a controlled rate freezer (e.g., Cryo-Med, Mt. Clemens, Mich.), then stored in the vapor phase of liquid nitrogen. Ten percent dimethylsulfoxide can be used as a cryoprotectant. After all collections from the patient have been made, CFU-GM containing cultures are thawed and pooled. The thawed cell collection is contacted with Flt3-ligand in combination with other cytokines listed above. Such exposure to Flt3-ligand will drive the CFU-GM to dendritic cell lineage. The dendritic cells are reinfused intravenously to the patient.

### Example 2

This example illustrates the ability of CD40L-stimulated dendritic cells to present allo-antigen and therefore cause proliferation of T cells. CD34+ cells were obtained from the bone marrow of a human donor, cultured for two weeks in the presence of one or more cytokines, such as GM-CSF, IL-4, FLT3-ligand, stem cell factor, as well as other cytokines known in the art that facilitate DC differentiation, and isolated by flow cytometry substantially as described in Example 1. Prior to their use in a mixed lymphocyte reaction (MLR), the dendritic cells were cultured for an additional 24 hours in the presence or absence of a soluble trimeric form of CD40L (1 μg/ml) in McCoy's enhanced media containing cytokines that support the growth of dendritic cells.

T cells were purified from the blood of a non-HLA matched donor by rosetting with 2-aminoethylisothiouronium bromide hydrobromide-treated sheep red blood cells. CD4+ and CD8+ populations were further purified using immunomagnetic selection using a MACS (Milenyi Biotec, Sunnyvale, Calif.) according to the manufacturer's protocol. Cell proliferation assays were conducted with the purified T cells in RPMI (10% heat-inactivated fetal bovine serum (FBS)), in the presence of titrated numbers of the dendritic cells, at 37° C. in a 10% CO₂ atmosphere. Approximately 1×10⁵ T cells per well were cultured in triplicate in round-bottomed 96-well microtiter plates (Coming) for seven days, in the presence of varying numbers of the unmatched dendritic cells. The cells were pulsed with 1 μCi/well of tritiated thymidine (25 Ci/nmole, Amersham, Arlington Heights, Ill.) for the final eight hours of culture.

Cells were harvested onto glass fiber discs with an automated cell harvester and incorporated cpm were measured by liquid scintillation spectrometry. The results demonstrated that three-fold fewer CD40L-activated dendritic cells were required to stimulate the equivalent proliferation of T cells compared to dendritic cells that had not been exposed to CD40L prior to their use in an MLR. This increase was most likely due to increased expression of cell surface molecules that stimulate allo-reactive T cells.

### Example 3

This example illustrates the ability of dendritic cells to stimulate antigen-specific proliferation of T cells. CD34+ cells were obtained from the bone marrow of a human donor believed to be reactive against tetanus toxoid, cultured for two weeks in the presence of selected cytokines, and isolated by flow cytometry substantially as described in Example 1. Prior to their use in a tetanus toxid (TTX) antigen presentation assay, the dendritic cells were cultured for an additional 24 hours in the presence or absence of a soluble trimeric form of CD40L (1 μg/ml ) in McCoy's enhanced media containing cytokines that support the growth of dendritic cells, then pulsed with purified TTX (Connaught Laboratory Inc., Swiftwater, Pa.), at 37° C. in a 10% CO₂ atmosphere for 24 hrs.

Autologous tetanus toxoid-reactive T cells were derived by culturing the CD34³¹ cells that were eluted from the CD34 antibody column in the presence of purified TTX and low concentrations of IL-2 and IL-7 (2 ng/ml and 5 ng/ml, respectively) for two weeks. The CD34⁻ population contains a percentage of T cells (about 5%), a proportion of which are reactive against tetanus toxoid, as well as other cell types that act as antigen presenting cells. By week 2, analysis of these cells indicated that they were about 90% T cells, the majority of which were tetanus toxoid-specific, with low levels of the T cell activation markers.

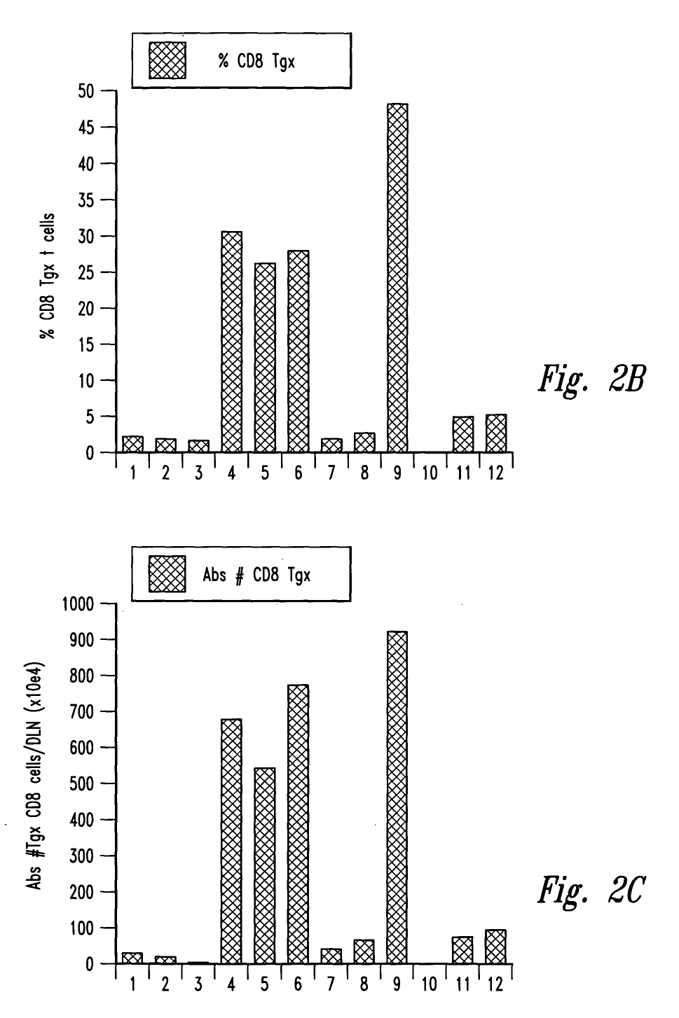

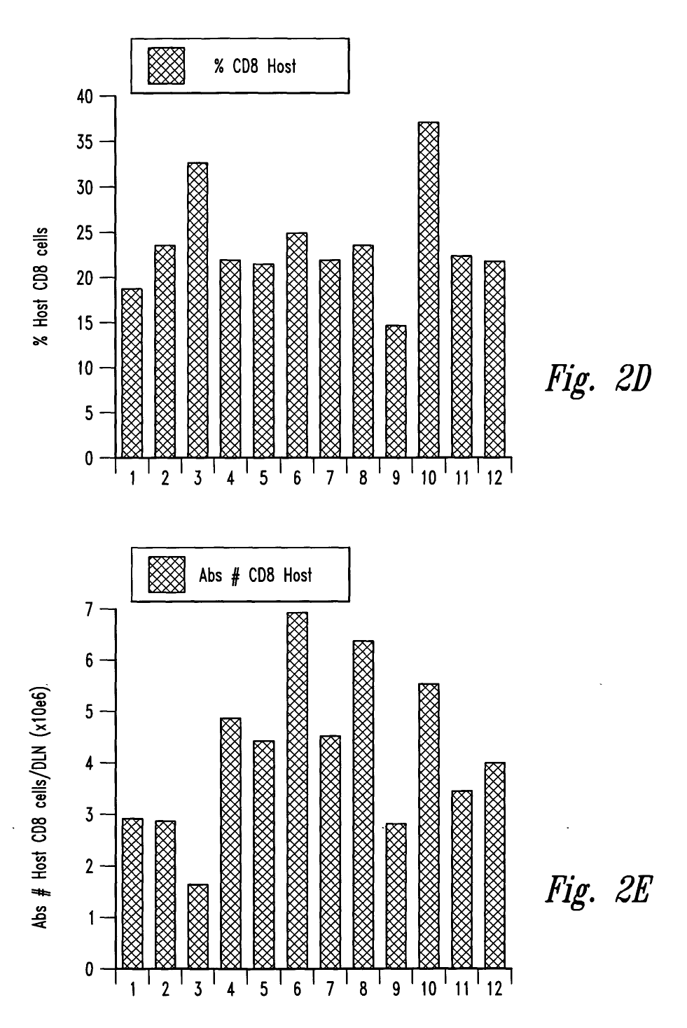

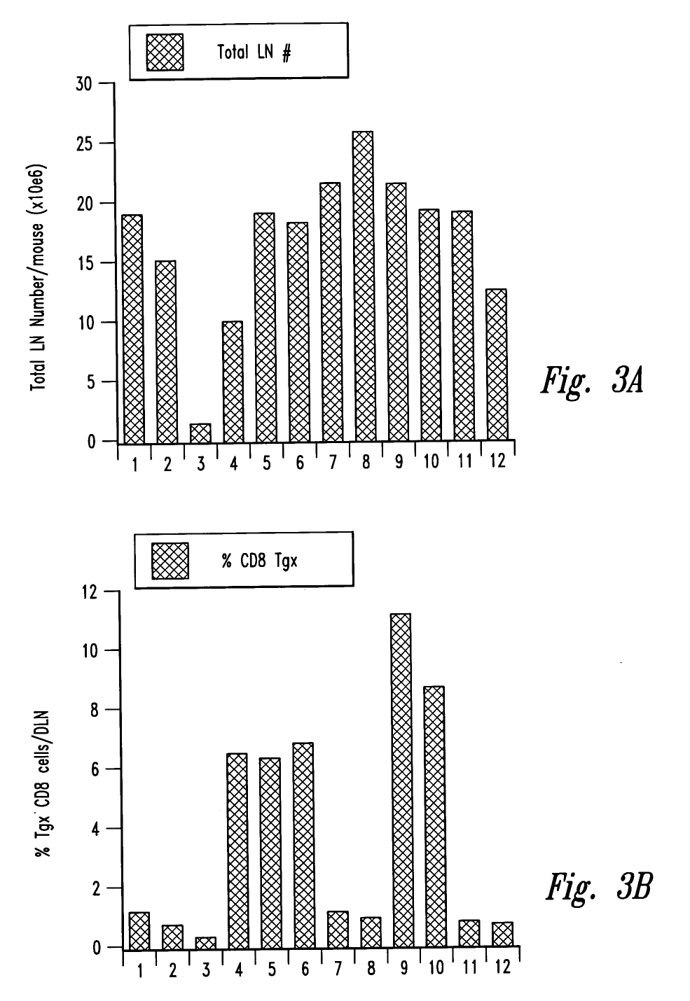

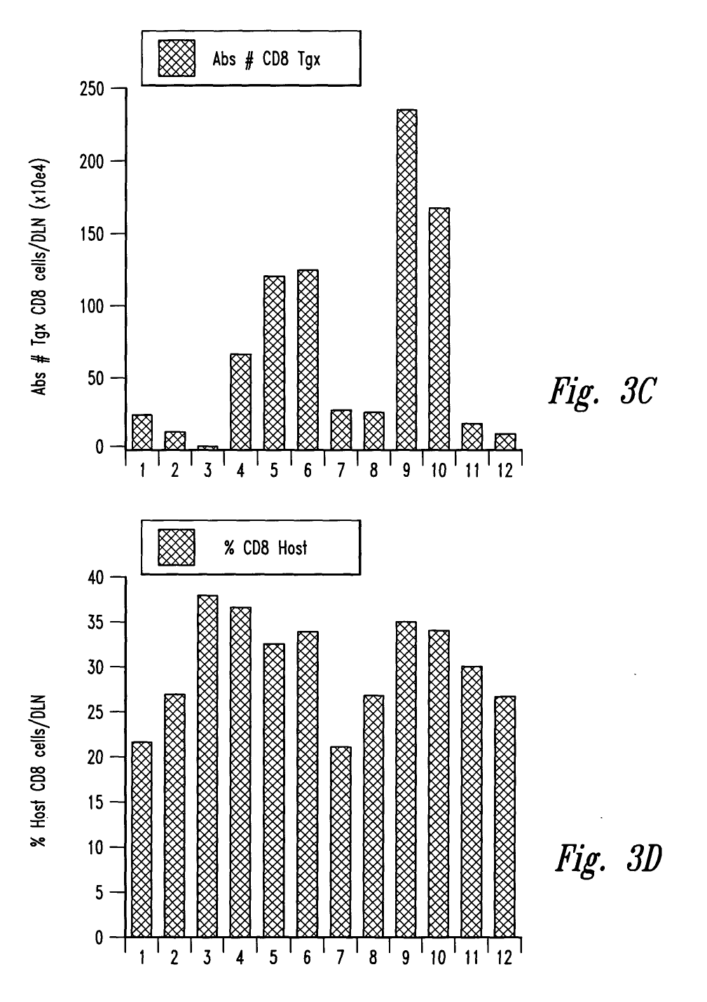

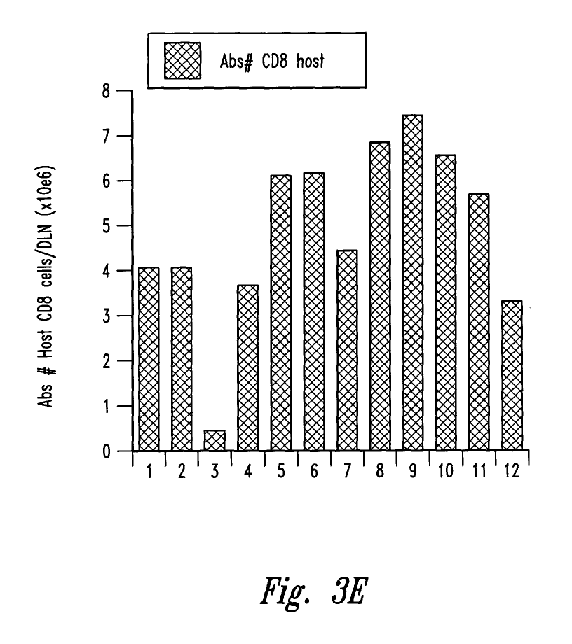

Antigen specific T cell proliferation assays were conducted with TTX-specific T cells from CD³⁴⁻ bone marrow cells as above, in RPMI with added 10% heat-inactivated fetal bovine serum (FBS), in the presence of the tetanus toxoid-pulsed dendritic cells, at 37° C. in a 10% CO₂ atmosphere. Approximately 1×10⁵ T cells per well were cultured in triplicate in round-bottomed 96-well microtiter plates (Coming) for five days, in the presence of a titrated number of dendritic cells. The cells were pulsed with 1 □Ci/well of tritiated thymidine (25 Ci/nmole, Amersham, Arlington Heights, Ill.) for the final four to eight hours of culture. Cells were harvested onto glass fiber discs with an automated cell harvester and incorporated cpm were measured by liquid scintillation spectrometry. The results, which are shown in FIG. 2, indicated that dendritic cells that are cultured with CD40L are about ten-fold less efficient at presenting antigen to TTX-specific T cells than dendritic cells that were not exposed to CD40L.

### Example 4

This Example describes a method for using Flt3-ligand to augment anti-tumor immune responses in vivo. Female C57BL/10J (B10) mice (The Jackson Laboratory, Bar Harbor, Me.) were injected with 5×10⁵ viable B10.2 or B10.5 fibrosarcoma tumor cells by intradermal injection in a midline ventral position in a total volume of 50 μl. The fibrosarcoma B10.2 and B10.5 lines are of B10 origin and have been described previously, see Lynch et a., Euro. J. Immunol., 21:1403 (1991) incorporated herein by reference. The fibrosarcoma B10.2 line was induced by subcutaneous implantation of a parrafin pellet containing 5 mg of methylcholanthrene, and the B10.5 line was induced by chronic exposure to ultraviolet radiation. The tumor cell lines were maintained in vitro in α-modified MEM containing 5% FBS, 2 nM L-glutamine, 50U/ml penicillin and 50 μg/ml streptomycin. Recombinant human Flt3-ligand (10 μg/injection) was administered on a daily basis over a 19-day period (unless otherwise noted) by subcutaneous injection in a total volume of 100 μl. Control mice were similarly injected with a similar volume of buffer containing 100 ng MSA. Tumor growth rates were determined by plotting the tumor size versus time after tumor challenge. Tumor size was calculated as the product of two perpendicular diameters, measured by calipers, and is expressed as the mean tumor size of only those mice bearing a tumor within a particular treatment group. The number of mice bearing tumors compared to the number challenged for each treatment group at the termination of an experiment are shown in the data below.

From Table I, the data is a compilation of six different experiments wherein tumor-bearing mice were either treated with Flt3-ligand or MSA. Complete tumor regression was observed in 19 of 50 Flt3-ligand treated mice compared to 1 of 30 in MSA-treated mice (p<0.0001 using Fishers Exact Test). The observation that the rate of tumor growth in Flt3-ligand treated mice (mean tumor size in tumor-bearing mice at week 5 post-tumor challenge was 60+/−8 mm²) was significantly reduced compared to MSA-treated mice (mean tumor size at week 5 post-tumor challenge was 185+/−17 mm²) was also confirmed (p.0001 by Analysis of Variance).

TABLE 2
Fibrosarcoma +/− Flt3-ligand Composite of Six Experiments
Tumor Size (mm²)

| Weeks Post | | | | |
|---|---|---|---|---|
| Tumor | MSA Control | Standard | Flt3-ligand | |
| Challenge | (100 ng/day) | Error | (10 μg/day) | Standard Error |
| 0 | 0 | 0 | 0 | 0 |
| 1 | 25 | 2.6 | 24 | 2.2 |
| 2 | 62 | 7.5 | 49 | 3.6 |
| 3 | 98 | 10.6 | 49 | 3.9 |
| 4 | 149 | 14.5 | 50 | 5 |
| 5 | 185 | 16.8 | 60 | 8.4 |

Tumor size was sharply retarded with Flt3-ligand compared to the control. Therefore, the data show that Flt3-ligand is an important cytokine in the augmentation of the immune response against foreign antigens, and in particular against cancer.

### Example 5

This Example demonstrates the use of Flt3-ligand in combination with interferon alpha to augment anti-tumor immune responses in vivo. In one study the B10.2 fibrosarcoma tumor cell line (described above) was implanted in C57BL/10J (B10) mice on day 0. One set of mice (n=10) was treated with recombinant human Flt3-ligand (50 μg/day, by subcutaneous injection) on days 10 to 29 post tumor challenge. Another set of mice (n=5) was treated with human interferon alpha (interferon alpha A/D; 60,000 U/day, by subcutaneous injection) on day 21 to 25 post tumor challenge. A third set of mice (n=5) was treated with Flt3-ligand on day 10 to 29 and also with interferon alpha on days 21 to 25. Control mice were injected with buffer containing 100 ng MSA. Tumor growth rates were determined by measuring tumor size over a 7-week period. Tumor size was calculated as the product of two perpendicular diameters, measured by calipers, and is expressed as the mean tumor size in mm². Only mice bearing tumors within each group were considered in determining the mean tumor size . The percent incidence of tumors was also determined (i.e., the number of mice bearing tumor compared to the number challenged) for each treatment group.

Table 3 shows the mean tumor size in tumor bearing animals, and Table 4 shows the percent incidence of tumors. This data demonstrates that interferon alpha synergizes with Flt3-ligand in enhancing immune response in the B 10.2 tumor model. Most significantly, the tumor rejection rate was 40% for Flt3-ligand alone and 80% for the combination of Flt3-ligand and interferon alpha.

TABLE 3
B10.2 Fibrosarcoma
Tumor size (mm²)

| | | Flt3-ligand | IFNα | |
|---|---|---|---|---|
| | MSA | (50 μg/day, | (60 K U/day, | Flt3-L + |
| Days Post | (100 ng/day) | days 1-20) | days 11-15) | IFNα |
| Tumor Challenge | (n = 10) | (n = 10) | (n = 5) | (n = 5) |
| 7 | 17 | 16 | 16 | 16 |
| 14 | 42 | 43 | 46 | 41 |
| 21 | 71 | 76 | 77 | 69 |
| 28 | 125 | 107 | 125 | 101 |
| 35 | 183 | 126 | 198 | 90 |
| 42 | 238 | 131 | 267 | 55 |
| 49 | 322 | 219 | 364 | 186 |

TABLE 4
B10.2 Fibrosarcoma
% Tumor incidence

| | | Flt3L | IFNα | |
|---|---|---|---|---|
| | MSA | (50 μg/day, | (60 K U/day, | |
| | (100 ng/day) | days 1-20) | days 11-15) | FL + IFNα |
| Days Post Tumor Challenge | (n = 10) | (n = 10) | (n = 5) | (n = 5) |
| 7 | 100 | 100 | 100 | 100 |
| 14 | 100 | 100 | 100 | 100 |
| 21 | 100 | 100 | 100 | 80 |
| 28 | 100 | 100 | 100 | 80 |
| 35 | 100 | 100 | 100 | 80 |
| 42 | 100 | 80 | 100 | 60 |
| 49 | 100 | 60 | 100 | 20 |

### Example 6

This Example demonstrates the use of Flt3-ligand in combination with a CD40 binding protein to augment anti-tumor immune responses in vivo. In one study C57BL/10J (B10) mice (The Jackson Laboratory, Bar Harbor, Me.) were injected intradermally with 5×10⁵ cells of the viable B10.2 fibrosarcoma tumor cell line described in Example 3 above, and the mice were subdivided into four sets each containing eight mice. In one set of mice, beginning on the same day as the tumor injections, recombinant human Flt3-ligand (10 μg/injection/day) was administered to each mouse on a daily basis over a 20-day period by subcutaneous injection in a total volume of 100 μl. In another set of mice each mouse was injected with the same volume and amount of CD40-L each day for 20 days. In a third set, each mouse was injected with a combination of 10 μg Flt3-ligand and 10 μg of CD40-L per day for 20 days. Control mice were similarly injected with a similar volume of buffer containing 100 ng MSA. Tumor growth rates were determined measuring tumor size each week after tumor challenge over a 6 week period. Tumor size was calculated as the product of two perpendicular diameters, measured by calipers, and is expressed as the mean tumor size. Only mice bearing tumors within each group were considered in determining the mean size. The frequency of tumor rejections was also determined and expressed as the number of mice bearing no tumors compared to the number challenged for each treatment group at the termination of an experiment.

Table 5 provides data in the form of mean tumor size in tumor bearing animals, calculated once a week over a 6 week period post challenge. Table 6 details the percent frequency of tumor rejection for each set of mice over a 6 week period post challenge. The data demonstrate that for tumor bearing mice, the mean tumor size mice in mice treated with Flt3-ligand and Flt3-ligand in combination with CD40-L is comparable and less than the tumor size in tumor bearing control mice. Significantly, however, mice receiving the combination therapy experienced significantly higher frequency of tumor rejection than mice receiving Flt3-ligand or CD40-L alone. More specifically, 6 weeks post challenge, 62.5% of the mice receiving the combination therapy experienced complete tumor rejection. By contrast, at 6 weeks post challenge, 25% of the mice receiving Flt3-ligand alone experienced complete tumor rejection and none of the mice receiving CD40-L alone or MSA experienced complete tumor rejection.

TABLE 5
B10.2 Fibrosarcoma
Tumor Size (mm²)

| Weeks | | | | |
|---|---|---|---|---|
| Post | | | | Flt3-L/CD40-L |
| Tumor | MSA Control | Flt3-L | CD40-L | (each |
| Challenge | (100 ng/day) | (10 μg/day) | (10 μg/day) | 10 μg/day) |
| 1 | 28 | 24 | 25 | 23 |
| 2 | 68 | 51 | 62 | 46 |
| 3 | 135 | 64 | 107 | 59 |
| 4 | 239 | 81 | 212 | 80 |
| 5 | 351 | 117 | 343 | 137 |
| 6 | 482 | 159 | 493 | 213 |

TABLE 6
B10.2 Fibrosarcoma
% Frequency of Tumor Rejection

| Weeks | | | | |
|---|---|---|---|---|
| Post | | | | Flt3-L/CD40-L |
| Tumor | MSA Control | Flt3-L | CD40-L | (each |
| Challenge | (100 ng/day) | (10 μg/day) | (10 μg/day) | 10 μg/day) |
| 1 | 0 | 0 | 0 | 0 |
| 2 | 0 | 0 | 0 | 0 |
| 3 | 0 | 0 | 0 | 0 |
| 4 | 0 | 12.5 | 0 | 50 |
| 5 | 0 | 25 | 0 | 62.5 |
| 6 | 0 | 25 | 0 | 62.5 |

In another study C3H/HeN mice were injected intradermally with 5×10⁵ cells of a very aggressive tumor, the 87 fibrosarcoma tumor cell line (generated by chronic exposure of C3H/HeN(MTV-) mice to ultraviolet radiation). The mice were then subdivided into four sets, each containing ten mice. In one set of mice, beginning the day after the tumor injections, recombinant human Flt3-ligand (10μg/injection/day) was administered to each mouse on a daily basis over a 20-day period by subcutaneous injection. In another set of mice, each mouse was injected with the same volume and amount of CD40-L each day, beginning at day 7 and continuing to day 20. In a third set, each mouse received a combination therapy of CD40-L and Flt3-ligand. The combination therapy included 10 μg/day of Flt3-ligand beginning the day after tumor injection and continuing until day 20 and 10 μg/day of CD40-L beginning at day 7 and continuing until day 20. Mice in a control group were similarly injected with a similar volume of buffer containing 100 ng MSA. Tumor growth rates were determined by measuring tumor size each week post tumor challenge over a 6 week period. Tumor size was calculated as the product of two perpendicular diameters, measured by calipers, and is expressed as the mean tumor size. Only mice bearing tumors were considered in determining the mean size. The frequency of tumor rejections was also determine and expressed as the number of mice bearing no tumors compared to the number challenged for each treatment group at the termination of an experiment.

Table 7 provides data in the form of mean tumor size in tumor bearing animals, calculated once a week over a 6 week period post challenge. Table 8 details the percent frequency of tumor rejection for each set of mice over a 6 week period post challenge. The data demonstrate that for tumor bearing mice, the mean tumor size mice in mice treated with Flt3-ligand in combination with CD40-L is significantly less than the tumor size in tumor bearing control mice and mice bearing tumors in the groups receiving only Flt3-ligand and only CD40-L. Significantly, mice receiving the combination therapy experienced significantly higher frequency of tumor rejection than mice receiving Flt3-ligand or CD40-L alone. More specifically, 6 weeks post challenge, 50% of the mice receiving the combination therapy experienced complete tumor rejection. By contrast, at 6 weeks post challenge, 10% of the mice receiving Flt3-ligand alone experienced complete tumor rejection and none of the mice receiving CD40-L alone or MSA experienced complete tumor rejection.

The observations described above demonstrate that a Flt3-ligand and CD40-L combination therapy can dramatically up-regulate anti-tumor immune responses in vivo. The data indicate that a synergy exists between Flt3-ligand and the CD40 binding protein, CD40-L, in that when used alone Flt3-ligand and CD40-L show little or no tumor rejection. In combination the rejection is dramatic. In addition to synergy, studies indicated that the combination of CD40-L and Flt3-ligand induced expression of IL-12 mRNA in the tumors.

TABLE 7
87 Fibrosarcoma
Tumor Size (mm²)

| Weeks | | | | |
|---|---|---|---|---|
| Post | | | | Flt3-L/CD40-L |
| Tumor | MSA Control | Flt3-L | CD40-L | (each |
| Challenge | (100 ng/day) | (10 μg/day) | (10 μg/day) | 10 μg/day) |
| 1 | 23 | 23 | 30 | 29 |
| 2 | 54 | 53 | 49 | 34 |
| 3 | 108 | 94 | 87 | 44 |
| 4 | 176 | 159 | 144 | 67 |
| 5 | 286 | 256 | 247 | 115 |
| 6 | 465 | 439 | 410 | 239 |

TABLE 8
87 Fibrosarcoma
% Frequency of Tumor Rejection

| Weeks | | | | |
|---|---|---|---|---|
| Post | | | | Flt3-L/CD40-L |
| Tumor | MSA Control | Flt3-L | CD40-L | (each |
| Challenge | (100 ng/day) | (10 μg/day) | (10 μg/day) | 10 μg/day) |
| 1 | 0 | 0 | 0 | 0 |
| 2 | 0 | 0 | 0 | 0 |
| 3 | 0 | 0 | 0 | 30 |
| 4 | 0 | 10 | 0 | 40 |
| 5 | 0 | 10 | 0 | 50 |
| 6 | 0 | 10 | 0 | 50 |

### Example 7

This Example demonstrates the use of Flt3-ligand in combination with an antibody reactive with 4-1BB to augment anti-tumor immune responses in vivo. In one study C57BL/10J (B10) mice (The Jackson Laboratory, Bar Harbor, Me.) were injected intradermally with 5×10⁵ cells of the viable B10.2 fibrosarcoma tumor cell line. In one set of mice, beginning on the same day as the tumor injections, recombinant human Flt3-L (10 μg/injection/day) was administered to each mouse on a daily basis over a 14-day period by subcutaneous injection in a total volume of 100 μl. In another set of mice each mouse was injected IP with 100 μg of rat anti mu 4-1BB (clone m6) on days 3 and 6 post tumor challenge. In a third set, each mouse was injected with 100 μg rat anti mu 4-1BB clone m6 on days 13 and 16. A fourth set of mice were injected with a combination of 10 μg Flt3-ligand on days 1-14 and 100 μg of rat anti mu 4-1BB clone m6 on days 13 and 16 post tumor challenge. Control mice were injected with buffer containing 100 ng MSA. Tumor growth rates were determined measuring tumor size each week after tumor challenge over a 5-week period. Tumor size was calculated as the product of two perpendicular diameters, measured by calipers, and is expressed as the mean tumor size in mm². Only mice bearing tumors within each group were considered in determining the mean tumor size. The percent incidence of tumors was also determine and expressed as the number of mice bearing tumors compared to the number challenged for each treatment group at the termination of an experiment.

Table 9 provides data in the form of mean tumor size in tumor bearing animals, calculated once a week over an 8 week period post challenge. Table 10 details the percent incidence of tumors for each set of mice over an 8 week period post challenge. The data demonstrate that for tumor bearing mice, the mean tumor size in mice treated with Flt3-ligand alone and the mean tumor size in mice treated with the anti 4-1BB regimen are similar. However, when Flt3-ligand in combination with an antibody reactive with 4-1BB is administered to mice, mean tumor size in tumor bearing mice is remarkably decreased. Specifically, at 5 weeks post tumor challenge, mice receiving the combination therapy had a mean tumor size of 0, indicating 100% tumor rejection. This data is supported by the numbers in Table 10 which demonstrate that mice receiving the combination therapy experienced significantly lower incidence of tumors than mice receiving Flt3-ligand or 4-1BB antibody alone. More specifically, at 5 weeks post challenge, all of the mice receiving the combination therapy experienced complete tumor rejection (0% tumor incidence). By contrast, at 5 weeks post challenge, 70% of the mice receiving Flt3-ligand alone had tumors and 50% and 70% of the mice receiving 4-1BB antibody alone had tumors. This data provides evidence that anti-4-1BB synergizes with Flt3-ligand in enhancing immune response.

TABLE 9
B10.2 Fibrosarcoma
Tumor Size (mm²)

| Weeks | MSA | | | | anti 4- |
|---|---|---|---|---|---|
| Post | Control | Flt3-L | anti 4-1BB | anti 4-1BB | 1BB(days 13 |
| Tumor | (100 ng/ | (10 μg/ | (100 μg, day | (100 μg days | and 16) and |
| Challenge | day) | day) | 3 and 6) | 13 and 16) | Flt3-L |
| 1 | 28 | 15 | 25 | 25 | 20 |
| 2 | 60 | 35 | 55 | 60 | 35 |
| 3 | 85 | 35 | 55 | 40 | 15 |
| 4 | 125 | 45 | 60 | 50 | 8 |
| 5 | 200 | 55 | 70 | 40 | 0 |
| 6 | 280 | 95 | 80 | 45 | 0 |
| 7 | | 125 | 130 | 115 | 0 |
| 8 | | | 160 | 135 | 0 |

TABLE 10
B10.2 Fibrosarcoma
% Tumor Incidence

| Weeks | MSA | | | | Flt3-L and |
|---|---|---|---|---|---|
| Post | Control | Flt3-L | anti 4-1BB | anti 4-1BB | anti 4-1BB |
| Tumor | (100 ng/ | (10 μg/ | (days 3 | (days 13 | (days 13 |
| Challenge | day) | day) | and 6) | and 16) | and 16) |
| 1 | 100 | 100 | 100 | 100 | 100 |
| 2 | 100 | 100 | 100 | 100 | 90 |
| 3 | 100 | 100 | 90 | 100 | 80 |
| 4 | 100 | 90 | 80 | 50 | 30 |
| 5 | 100 | 70 | 70 | 50 | 0 |
| 6 | 100 | 60 | 60 | 40 | 0 |
| 7 | 100 | 60 | 50 | 20 | 0 |
| 8 | 100 | 60 | 50 | 20 | 0 |

### Example 8

This example describes one embodiment of a Flt3-ligand immunization protocol that has been shown to enhance antigen-specific immune responses. The experiments were designed to determine whether Flt3-ligand, pegylated GM-CSF and CD40L enhance immune responses to vaccines.

The following studies used the well-known OT-I and OT-II transgenic mouse models. The studies involve the intravenous transplantation of small numbers of T-cells from the OT-I and OT-II transgenic mice into non-transgenic congenic mice. The transgenic mice express T-cell receptors (TCR) that specifically recognize select peptides derived from chicken egg ovalbumin (OVA) in the context of MHC class I and II. OT-I mice, which have a C57BL/6 background, are transgenic for the α- and β-chains of a TCR that is specific for the OVA₂₅₇₋₂₆₄ peptide (SIINFEKL—SEQ ID NO:3) bound to H-2K^{b} (Hogquist, K. A., et al., Cell, 76, 17-27, 1994). OT-II mice, which also have a C57BL/6 background, are transgenic for a TCR that is specific for the OVA₃₂₃-₃₃₉ peptide (ISQAVHAAHAEINEAGR—SEQ ID NO:4) presented in the context of IA^{b} (Barnden, M. J., et al., Immunol. Cell Biol., 76, 34, 1998).

Approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with 25 ug each of the OT-1 and OT-II peptides emulsified in Incomplete Freund's Adjuvant (IFA—Difco/Becton Dickinson, Franklin Lakes, N.J.). IFA is a paraffin oil and is thought to serve as a depot for the peptides, as well as a pro-inflammatory signal.

The treatment groups are presented in Table 11. Flt3-ligand was produced by well-known recombinant DNA technology in a Chinese hamster ovary (CHO) cell line and is available from Immunex Corporation, Seattle, Wash. Group 1 was immunized with ova peptides formulated in Complete Freund's Adjuvant (CFA) at day 11, which served as a positive control. Group 2 received ova peptides formulated in Incomplete Freund's Adjuvant (IFA) on day 11. Group 3 received ova peptides formulated in phosphate buffered saline (PBS) on day 11. Group 4 received 10 ug Flt3-ligand s.c. per day at the nape of the neck for ten consecutive days and immunized with ova peptides formulated in IFA on day 11. Group 5 received 10 ug Flt3-ligand s.c. per day at the nape of the neck for ten consecutive days and immunized with ova peptides formulated in IFA on day 11 and received 10 ug CD40L s.c. at the site of immunization on days 11 and 12. Group 6 received 10 ug Flt3-ligand s.c. per day at the nape of the neck for ten consecutive days and immunized with ova peptides formulated in IFA on day 11 and received 10 ug CD40L s.c. at a site distal to the site of immunization (nape of neck) on days 11 through 15. Group 7 was immunized with ova peptides formulated in IFA on day 11 and received 10 ug CD40L s.c. at the site of immunization on days 11 and 12. Group 8 was immunized with ova peptides formulated in IFA on day 11 and received 10 ug CD40L s.c. at a site distal to the site of immunization (nape of neck) on days 11 through 15. Group 9 received 10 ug Flt3-ligand s.c. at the nape of the neck per day for ten consecutive days and immunized with ova peptides formulated in IFA on day 11 and received 5 ug PEGylated GM-CSF (pGM-CSF) s.c. at the site of immunization on days 11 and 12. Group 10 received 10 ug Flt3-ligand s.c. per day at the nape of the neck for ten consecutive days and immunized with ova peptides formulated in IFA on day 11 and received 5 ug PEGylated GM-CSF (pGM-CSF) s.c. at a site distal to the site of immunization (nape of neck) on days 11 through 15. Group 11 was immunized with ova peptides formulated in IFA on day 11 and received 5 ug pGM-CSF s.c. at the site of immunization on days 11 and 12. Group 12 was immunized with ova peptides formulated in IFA on day 11 and received 5 ug pGM-CSF s.c. at a site distal to the site of immunization (nape of neck) on days 11 through 15.

TABLE 11

| Grp | | Animals | Animals |
|---|---|---|---|
| # | Treatment Groups | per group | per time pt. |
| 1 | CFA + peptides | 8 | 3/3/2 |
| 2 | IFA + peptides | 8 | 3/3/2 |
| 3 | PBS + peptides | 8 | 3/3/2 |
| 4 | Flt3-L d1-10, imm. d11 | 8 | 3/3/2 |
| 5 | Flt3-L d1-10, imm. d11 + CD40L at imm. site d11 & 12 | 8 | 3/3/2 |
| 6 | Flt3-L d1-10, imm. d11 + CD40L at distal site d11-15 | 8 | 3/3/2 |
| 7 | CD40L at imm. site d11 & 12 | 8 | 3/3/2 |
| 8 | CD40L at distal site d11-15 | 8 | 3/3/2 |
| 9 | Flt3-L d1-10, imm. d11 + pGM-CSF at imm. site d11 & 12 | 8 | 3/3/2 |
| 10* | Flt3-L d1-10, imm. d11 + pGM-CSF at distal site d11-15 | 8 | 3/3/2 |
| 11 | pGM-CSF at imm. site d11 & 12 | 8 | 3/3/2 |
| 12 | pGM-CSF at distal site d11-15 | 8 | 3/3/2 |

*Group 10: mice for day 5 did not receive transgenic cells, but were cytokine-treated and immunized, as such, they are not be considered in the analysis below.

On days 2, 5 and 9 post immunization, cells from the draining lymph nodes were harvested (typically from lymph nodes that drain the immunization site: two inguinal and one accesory axillary). A single cell suspension prepared, and the frequency of donor CD4+ and CD8+ transgenic T cells were calculated by flow cytometry analysis (FACs). Functional assays were also performed in standard cytotoxic T cell assays which measured the specific lysis of class I peptide-pulsed target cells, as well as by IFNγ production after in vitro restimulation with OT-II peptide, as measured by ELISPOT™ (Becton Dickinson, Franklin Lakes, N.J.).

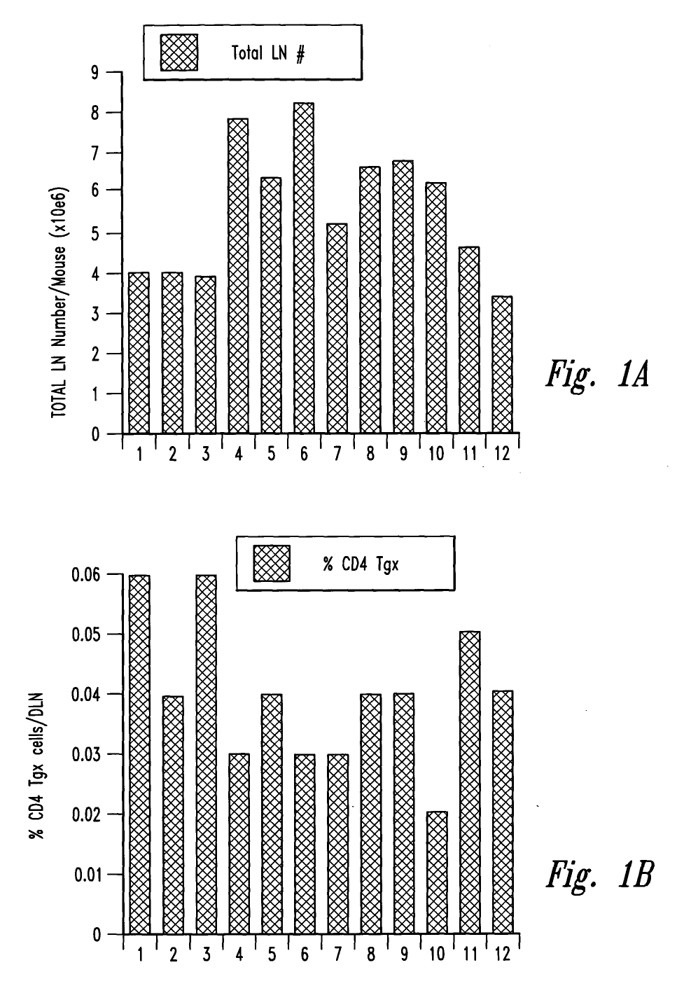

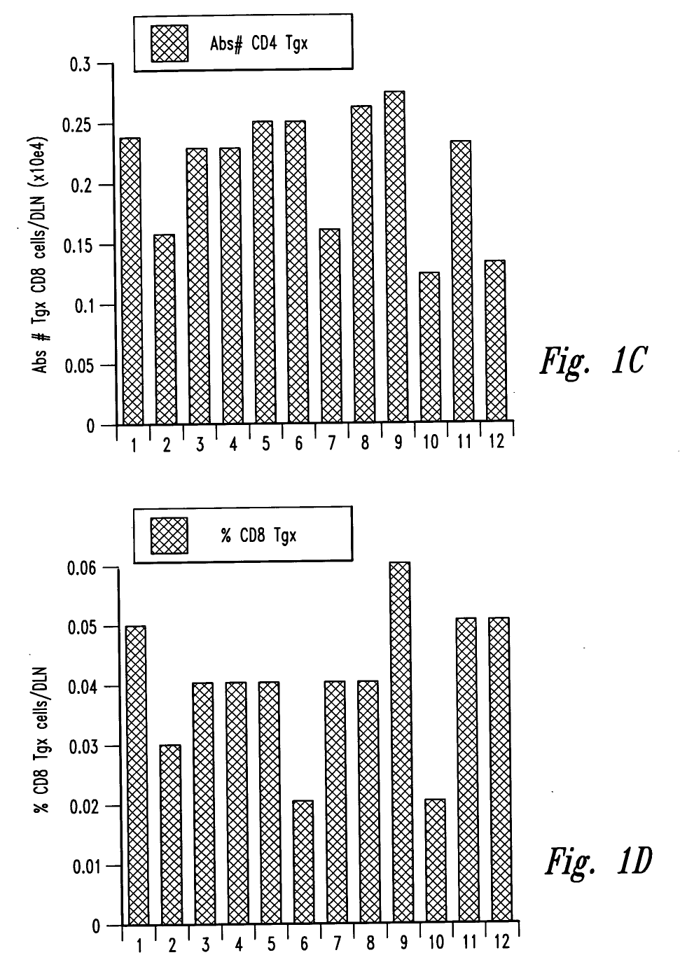

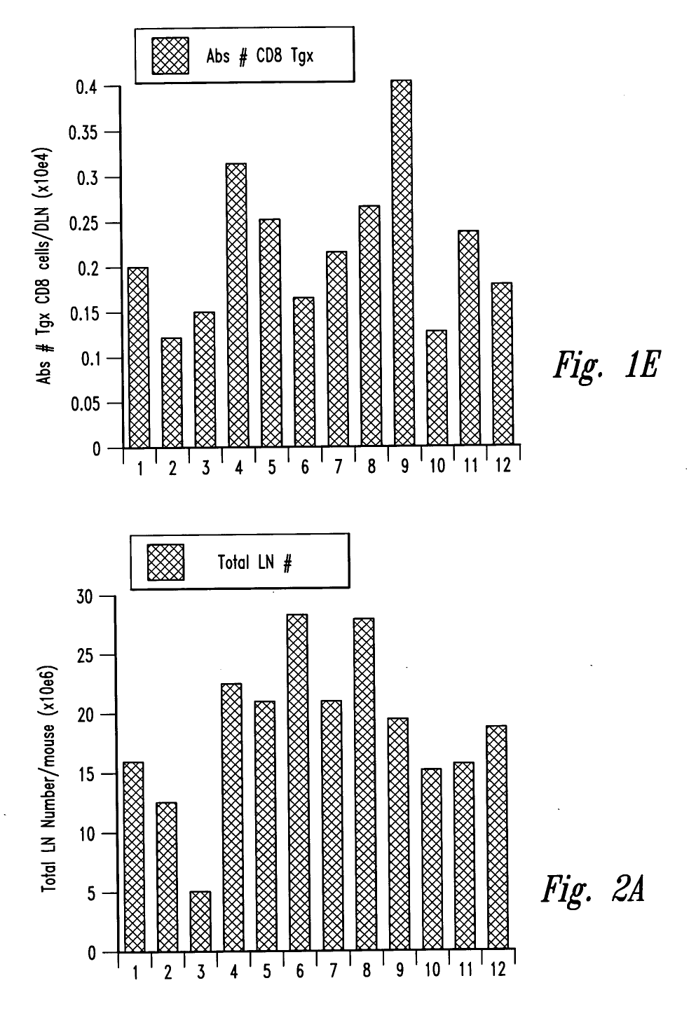

As shown in FIGS. 1A-E, 2A-E and 3A-E, a dramatic expansion of CD8+ transgenic T cells occurred when mice were pre-treated with Flt3-ligand. On day 5 post immunization, the number of transgenic CD8+ T cells in the draining lymph nodes (DLN) was approximately 34 times higher in mice receiving Flt3-ligand prior to immunization with peptides formulated in IFA over mice not receiving Flt3-ligand, and 114-fold higher than mice only receiving peptides formulated in PBS. The further addition of pGM-CSF post immunization appears to augment and prolong CD8+ T cell expansion induced by Flt3-ligand.

The combination of Flt3-ligand pre-treatment and immunization with antigen formulated in IFA adjuvant had a dramatic effect on CTL generation. The CD8+ T cells expanded by the Flt3-ligand immunization protocol were functional, antigen-specific effector cells as measured by standard CTL assays after 5 days in vitro restimulation with OT-I peptide-pulsed targets (conventional protocol for measuring CTL activity). Five days after immunization, antigen-specific CTL had expanded to comprise 25-40% of all cells in the DLN, equal to about 2.5-9×10⁶ cells (n=4 experiments). FIGS. 4A and 4B show that the groups receiving Flt3-ligand prior to immunization, and optionally receiving an auxiliary molecule, such as pGM-CSF or CD40-L, had the highest levels of antigen-specific CTL activity.

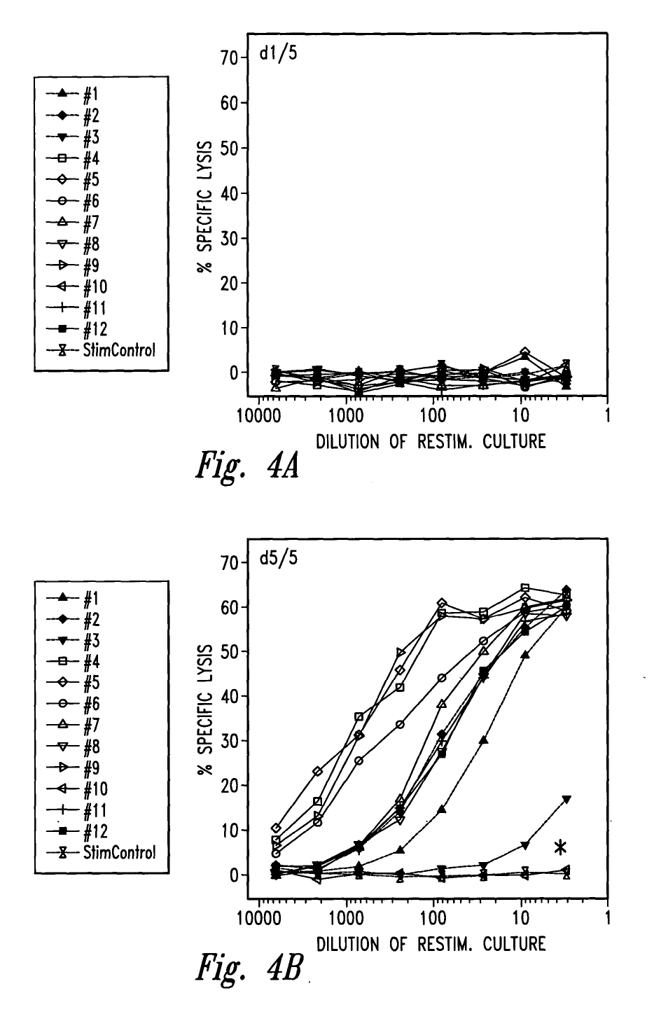

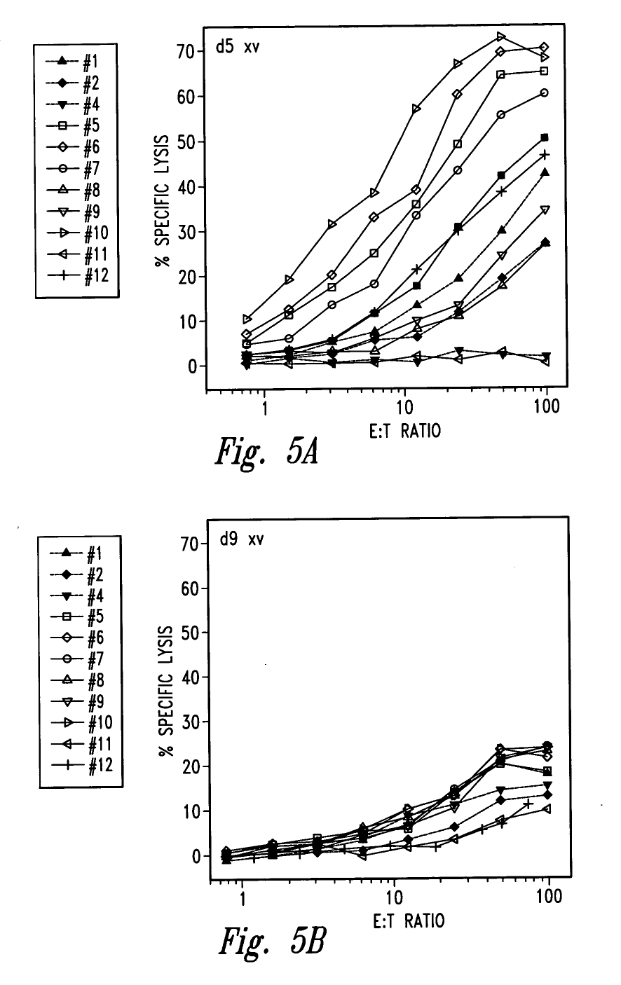

Remarkably, groups receiving Flt3-ligand prior to immunization, and optionally receiving an auxiliary molecule, such as pGM-CSF or CD40-L, had such a heightened immune response that CTL activity was measured in T-cell populations isolated directly from the DLN, i.e., there was no requirement for in vitro restimulation to uncover antigen-specific CTL activity (see FIGS. 5A and 5B). More specifically, the CTL assays were performed as follows. CTL activity was measured in a standard ⁵¹Cr-release assay. C1498 (H-2^{b}) target cells were pulsed with 50 μCi ⁵¹Cr (Amersham Biosciences, Piscataway, N.J.) per 1×10⁶ cells, in complete RPMI medium, for 1 hour at 37° C. with or without 1 μM OTI peptide (SIINFEKL, Immunex). Labeled target cells were washed four times and 1×10⁴ cells were added to serial titrations of DLN cells (effectors). Effector: target ratios ranged from 100:1 to 0.78:1. Assays were performed in complete RPMI medium containing RPMI-1640 (JRH Biosciences, Lenexa, Kans.) supplemented with 10% heat-inactivated fetal bovine serum (Gibco Invitrogen, Carlsbad, Calif.), 100 μM MEM non-essential amino acids (Gibco), 1 mM MEM sodium pyruvate (Gibco), 55 μM 2-mercaptoethanol (Gibco), 50 U/ml penicillin (Calbiochem, San Diego, Calif.), 50 μg/ml streptomycin (Mediatech, Herndon, Va.), and 2 mM L-glutamine (JRH Biosciences). After a 6-hour incubation at 37° C., 25 μl of cell-free supernatant was removed and transferred to a Packard LumaPlate (Packard BioScience, Meriden, Conn.). Plates were read for 60 seconds per well on the Packard TopCount (Hewlett Packard, Palto Alto, Calif.). Spontaneous and maximum chromium release were determined by the addition of assay medium or 0.1% Triton X-100 (Pierce Chemical Company, Rockford, Ill.) to target cells, respectively. Percent specific lysis was calculated as 100×(experimental release cpm−spontaneous release cpm)/(maximum release cpm−spontaneous release cpm).

These results demonstrate that the antigen-specific CD8+ CTL immune response generated in mice pre-treated with Flt3-ligand and subsequently vaccinated were as potent as those generated in an acute response to a viral infection. Taken together, these results demonstrate that Flt3-ligand immunization protocols increase both the magnitude and duration of antigen-specific effector cell responses.

### Example 9

These studies were performed to determine the role of T-helper cells in Flt3-ligand immunization protocols. In this embodiment, 10 ug Flt3-ligand was administered subcutaneously per day at the nape of the neck for ten consecutive days. Approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with 25 ug of the OT-1. As described in more detail below, some groups receivedOT-II peptides emulsified in Incomplete Freund's Adjuvant (IFA) or PBS or keyhole limpet hemocyanin (KLH) (Calbiochem, San Diego Calif.) to facilitate T-helper responses.

The treatment groups for this study are presented in Table 12 below. Group 1 was immunized with OT-I and OT-II peptides formulated in IFA at day 11, which served as a positive control. Group 2 received OT-I and OT-II peptides formulated in phosphate buffered saline (PBS) on day 11. Group 3 received 10 ug Flt3-ligand s.c. per day at the nape of the neck for ten consecutive days and immunized on day 11 with OT-I and OT-II peptides formulated in IFA. Group 4 received 10 ug Flt3-ligand s.c. per day at the nape of the neck for ten consecutive days and immunized on day 11 with OT-I peptides only formulated in IFA. Group 5 received 10 ug Flt3-ligand s.c. per day at the nape of the neck for ten consecutive days and immunized on day 11with OT-I peptides and 25 ug KLH formulated in IFA. Group 6 received 10 ug Flt3-ligand s.c. per day at the nape of the neck for ten consecutive days and immunized on day 11 with OT-I and OT-II peptides formulated in PBS.

TABLE 12

| Grp | | Animals | Animals |
|---|---|---|---|
| # | Treatment Groups | per group | per time pt. |
| 1 | IFA + TO-I & II peptides | 8 | 3/3/2 |
| 2 | PBS + TO-I & II peptides | 9 | 3/3/3 |
| 3 | Flt3-L d1-10, imm. d11 with TO-I & II peptides in IFA | 9 | 3/3/3 |
| 4 | Flt3-L d1-10, imm. d11 with TO-I peptide in IFA | 9 | 3/3/3 |
| 5 | Flt3-L d1-10, imm. d11 with TO-I peptide + KLH in IFA | 9 | 3/3/3 |
| 6 | Flt3-L d1-10, imm. d11 with TO-I & II peptides in PBS | 9 | 3/3/3 |

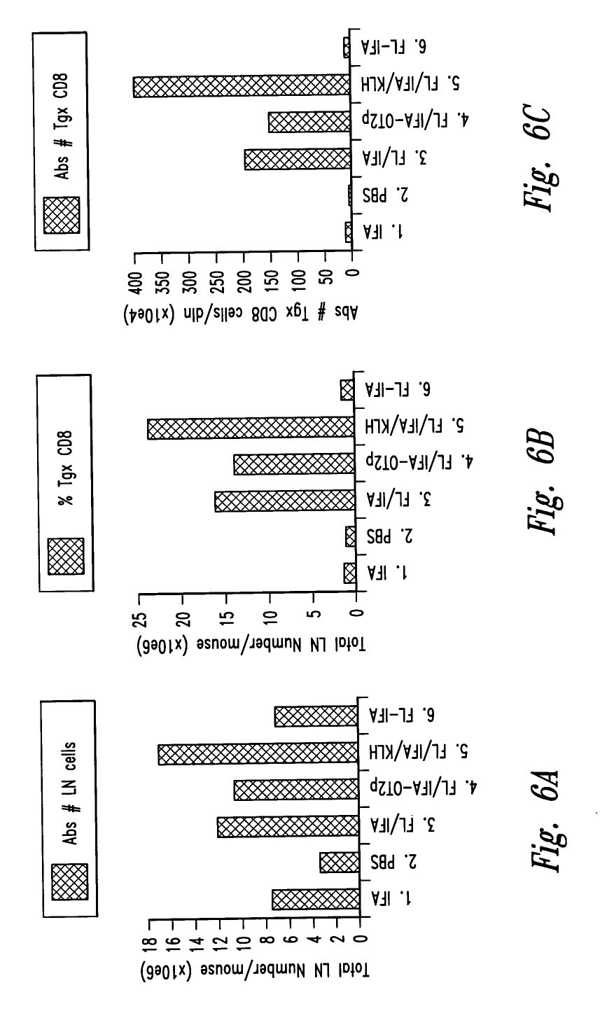

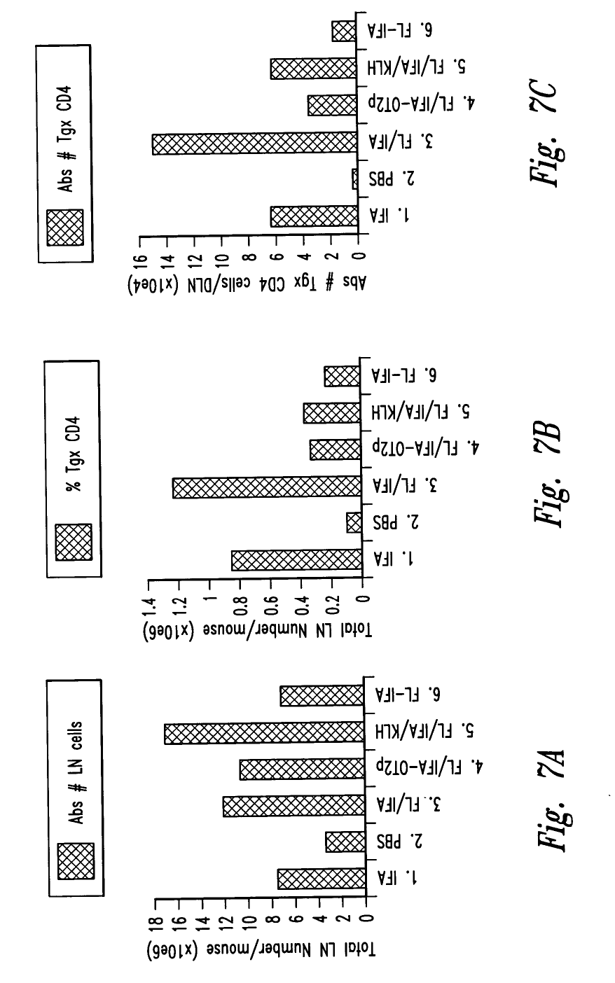

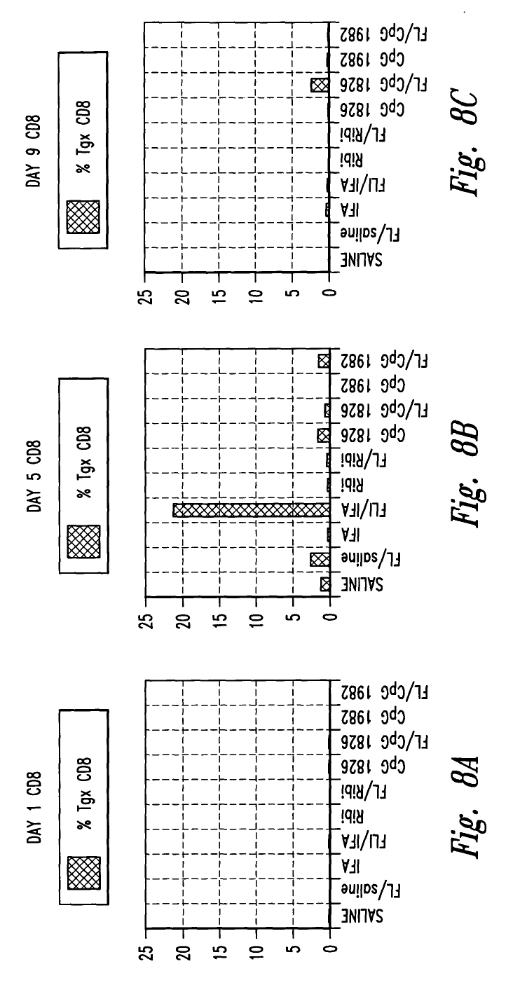

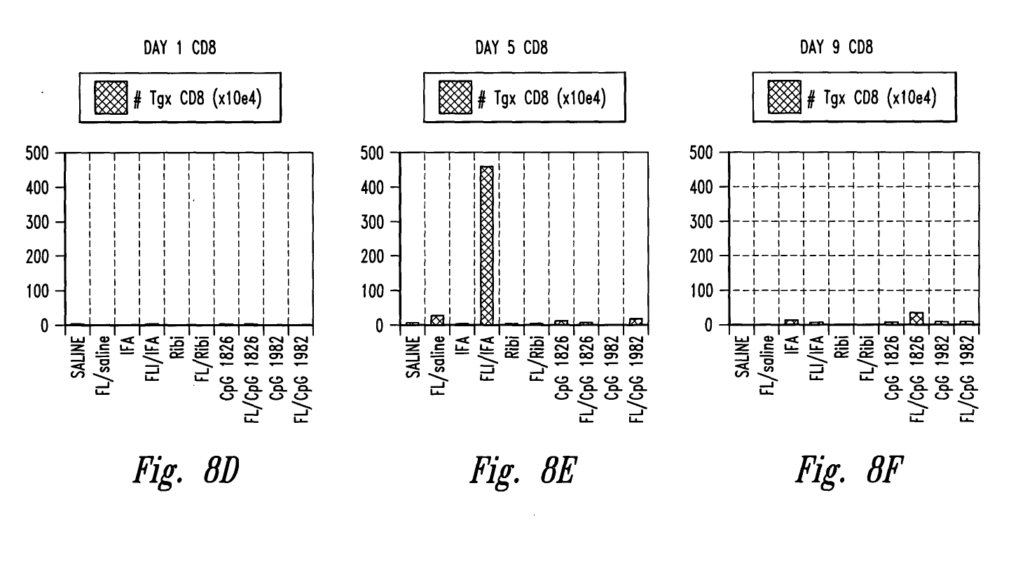

As in previous studies, the combination of Flt3-ligand pre-treatment and immunization with antigen formulated in IFA had a dramatic effect on antigen-specific CD8+ CTL generation. The inclusion of KLH as a substitute helper protein did elicit a surprising increase in the percent transgenic CD8+ cells (FIGS. 6A-6C). Therefore, proteins having MHC-II epitopes that increase the number of IL-2 producing T-helper cells, which in turn enhance CD8+ CTL responses may be included in Flt3-ligand immunization protocols as an additional antigen in the vaccine formulation. FIGS. 7A-7C show that the group 3, which received Flt3-ligand prior to immunization had dramatically higher levels of transgenic CD4+ T cells. Functional studies have shown that antigen-specific, biologically functional T-helper cells are generated, as measured by standard proliferation assays and IFNγ production (measured by standard ELISA techniques.

### Example 10

These experiments compared various adjuvants in Flt3-ligand immunization protocols. The adjuvants tested included: IFA (Incomplete Freund's Adjuvant), MPL™ (3-Q-desacyl-4′-monophosphoryl lipid A-Ribi/Corixa Corp., Seattle, Wash.), CpG 1826 and 1982 (oligonucleotides containing unmethylated CpG nucleotides that mimic bacteria and/or viruses, see, J. Immunol. 2000 164:1617), Alum (aluminum hydroxide) and Quil-A (Quil-A saponin—extracted from the bark of Quillaja saponaria, which is the active component of Immune Stimulating Complexes-ISCOMS). As before, 10 ug Flt3-ligand was administered subcutaneously per day at the nape of the neck for ten consecutive days. Approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with the OT-1 and OT-II peptides (25 ug each) formulated/mixed with either: PBS, IFA, MPL™, CpG 1826 in PBS or CpG 1982 in PBS.

The treatment groups for this study are presented in Table 13. Group 1 was immunized with OT-I and OT-II peptides formulated in PBS at day 11. Group 2 received 10 ug Flt3-ligand s.c. per day at the nape of the neck for ten consecutive days and immunized on day 11 with OT-I and OT-II peptides formulated in PBS. Group 3 was immunized with OT-I and OT-II peptides formulated in IFA at day 11. Group 4 received 10 ug Flt3-ligand s.c. per day at the nape of the neck for ten consecutive days and immunized on day 11 with OT-I and OT-II peptides formulated in IFA. Group 5 was immunized with OT-I and OT-II peptides formulated in MPL™ at day 11. Group 6 received 10 ug Flt3-ligand s.c. per day at the nape of the neck for ten consecutive days and immunized on day 11 with OT-I and OT-II peptides formulated in MPL™. Group 7 was immunized with OT-I and OT-II peptides mixed with CpG (1826) in PBS at day 11. Group 8 received 10 ug Flt3-ligand s.c. per day at the nape of the neck for ten consecutive days and immunized on day 11 with OT-I and OT-II peptides mixed with CpG (1826) in PBS. Group 9 was immunized with OT-I and OT-II peptides mixed with CpG (1982) in PBS at day 11. Group 10 received 10 ug Flt3-ligand s.c. per day at the nape of the neck for ten consecutive days and immunized on day 11 with OT-I and OT-II peptides mixed with CpG (1982) in PBS.

TABLE 13

| Grp | | Animals | Animals |
|---|---|---|---|
| # | Treatment Groups | per group | per time pt. |
| 1 | TO-I & II peptides in PBS | 9 | 3/3/3 |
| 2 | Flt3-L d1-10, imm. d11 with TO-I & II peptides in PBS | 9 | 3/3/3 |
| 3 | TO-I & II peptides in IFA | 9 | 3/3/3 |
| 4 | Flt3-L d1-10, imm. d11 with TO-I & II peptides in IFA | 9 | 3/3/3 |
| 5 | TO-I & II peptides in MPL ™ | 9 | 3/3/3 |
| 6 | Flt3-L d1-10, imm. d11 with TO-I & II peptides in MPL ™ | 9 | 3/3/3 |
| 7 | TO-I & II peptides in CpG(1826)/PBS | 9 | 3/3/3 |
| 8 | Flt3-L d1-10, imm. d11 with TO-I & II peptides in | 9 | 3/3/3 |
| | CpG(1826)/PBS | | |
| 9 | TO-I & II peptides in CpG(1982)/PBS | 9 | 3/3/3 |
| 10 | Flt3-L d1-10, imm. d11 with TO-I & II peptides in | 9 | 3/3/3 |
| | CpG(1982)/PBS | | |

MPL ™ (Corixa Corp., Seattle, WA); (IFA - Difco/Bectin Dickinson, Franklin Lakes, NJ)

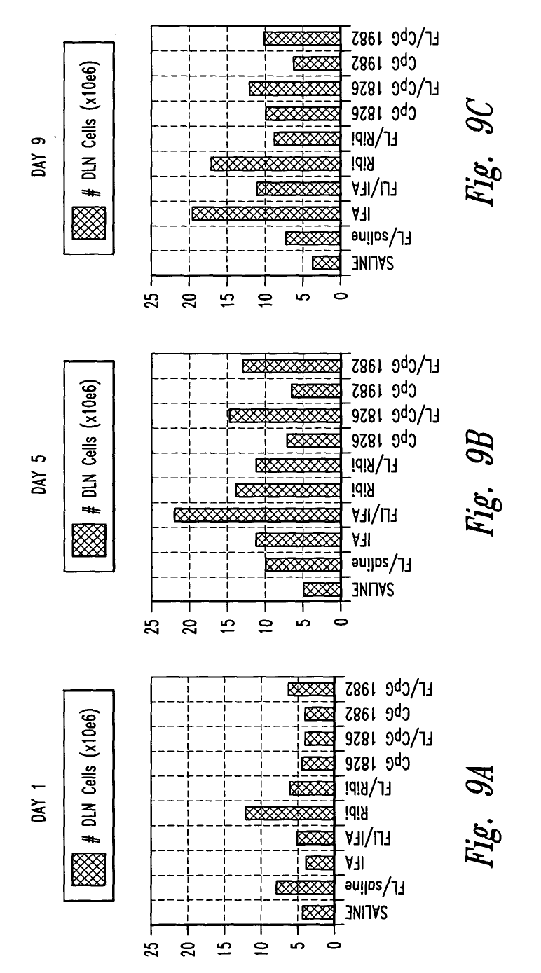

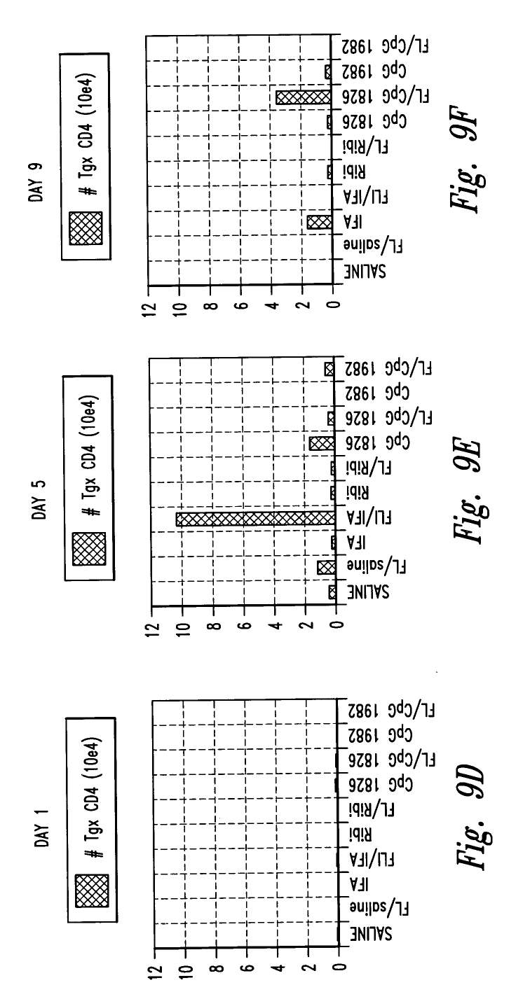

As shown in FIGS. 8B and 8E, the combination of Flt3-ligand pre-treatment and immunization with antigen formulated in IFA generated a tremendous antigen-specific CD8+CTL response in comparison to OT-I & II peptides formulated/mixed with MPL™ and CpG sequences. As with previous studies, the CTL from the Flt3-ligand/IFA group displayed antigen-specific cytolytic activity in T-cell populations isolated directly from the draining lymph nodes. Furthermore, this group had higher levels of transgenic CD4+ T cells (FIG. 9E). Functional studies showed that these CD4+ T cells displayed antigen-specific proliferation when cultured in the presence of OT-II peptide-pulsed targets as well as IFNγ production (measured by ELISA). In addition, the group receiving Flt3-ligand pre-treatment and immunization with antigen mixed with CpG (1826)/PBS showed an increase in the number of transgenic CD4+ T cells over the group not receiving Flt3-ligand pretreatment, as well as weak OT-II peptide-induced IFNγ production. This data suggests that the combination of Flt3-ligand pretreatment and different adjuvants may preferentially influence different arms of the immune system and also influence the immune system in a temporally-based manner.

A similar study was conducted to compare IFA, Alum, and Quil-A in Flt3-ligand immunization protocols. As before, 10 ug Flt3-ligand was administered subcutaneously per day at the nape of the neck for ten consecutive days. Approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with the OT-1 and OT-II peptides (25 ug each) formulated/mixed with either: IFA, Alum or Quil-A.

The treatment groups for this study are presented in Table 14. Group 1 received 10 ug Flt3-ligand s.c. per day at the nape of the neck for ten consecutive days and immunized on day 11 with OT-I and OT-II peptides formulated in PBS. Group 2 was immunized with OT-I and OT-II peptides formulated in IFA at day 11. Group 3 received 10 ug Flt3-ligand s.c. per day at the nape of the neck for ten consecutive days and immunized on day 11 with OT-I and OT-II peptides formulated in IFA. Group 4 was immunized with OT-I and OT-II peptides formulated in Alum at day 11. Group 5 received 10 ug Flt3-ligand s.c. per day at the nape of the neck for ten consecutive days and immunized on day 11 with OT-I and OT-il peptides formulated in Alum. Group 6 was immunized with OT-I and OT-II peptides formulated in Quil-A at day 11. Group 7 received 10 ug Flt3-ligand s.c. per day at the nape of the neck for ten consecutive days and immunized on day 11 with OT-I and OT-II peptides formulated in Quil-A.

TABLE 14

| Grp | | Animals | Animals |
|---|---|---|---|
| # | Treatment Groups | per group | per time pt. |
| 1 | Flt3-L d1-10, imm. d11 with TO-I & II peptides in PBS | 9 | 3/3/3 |
| 2 | TO-I & II peptides in IFA | 9 | 3/3/3 |
| 3 | Flt3-L d1-10, imm. d11 with TO-I & II peptides in IFA | 9 | 3/3/3 |
| 4 | TO-I & II peptides in Alum | 9 | 3/3/3 |
| 5 | Flt3-L d1-10, imm. d11 with TO-I & II peptides in Alum | 9 | 3/3/3 |
| 6 | TO-I & II peptides in Quil-A | 9 | 3/3/3 |
| 7 | Flt3-L d1-10, imm. d11 with TO-I & II peptides in Quil-A | 9 | 3/3/3 |

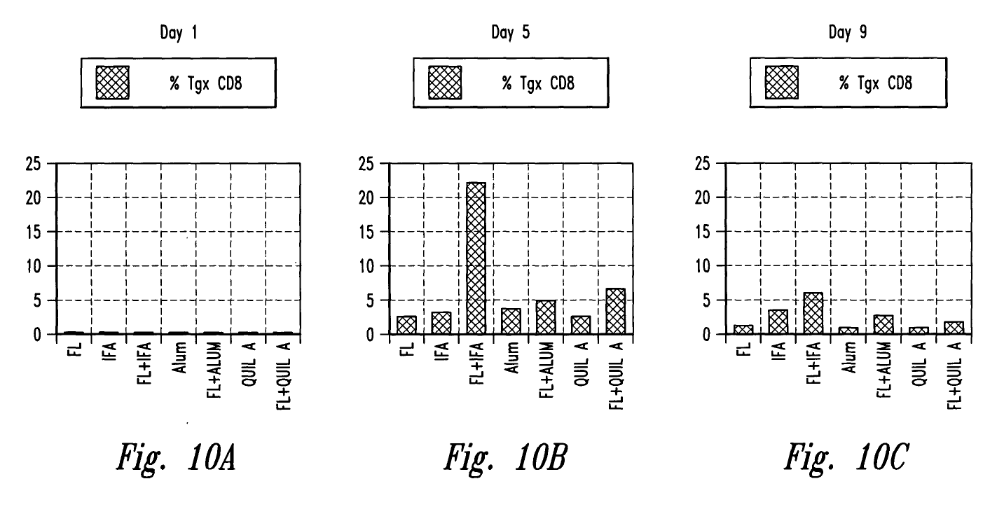

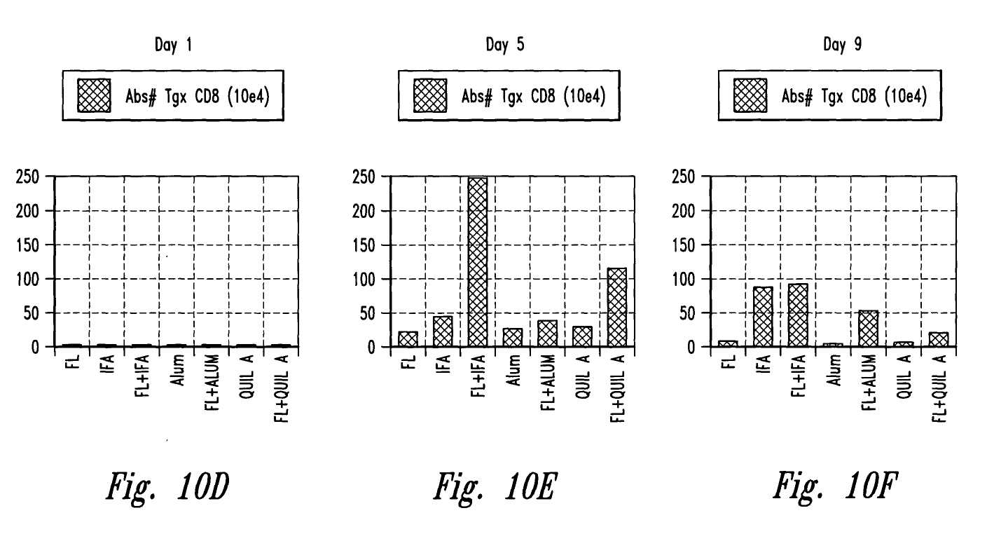

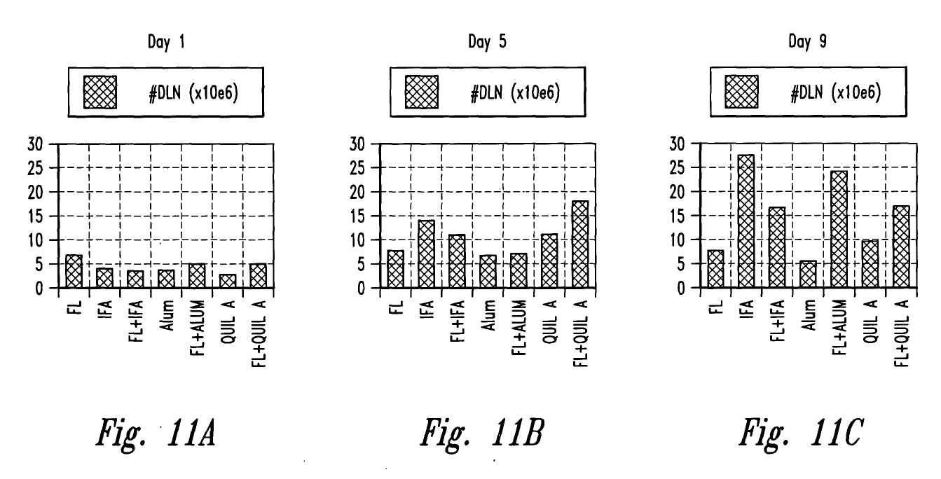

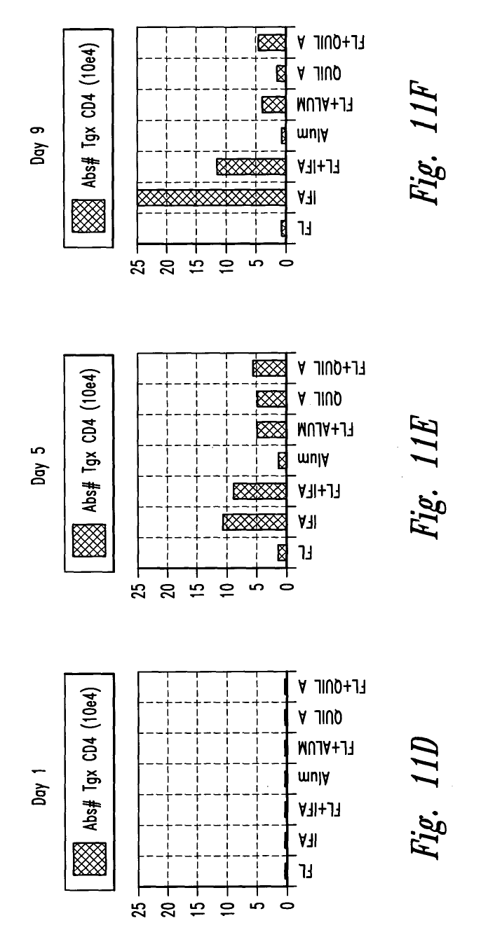

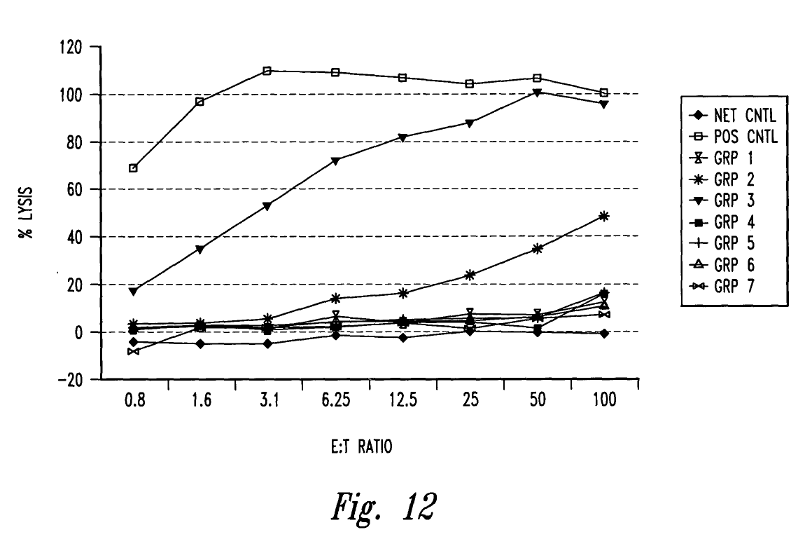

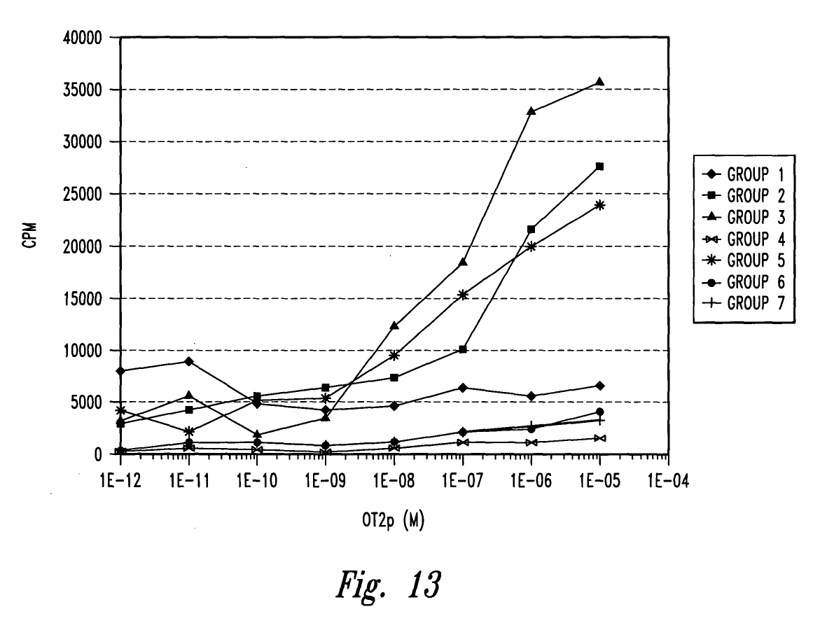

Consistent with previous studies, the combination of Flt3-ligand pretreatment and immunization with antigen formulated in IFA generated a markedly enhanced antigen-specific CD8+ CTL response (FIGS. 10B and 10E). The CTL from the Flt3-ligand/IFA group displayed antigen-specific cytolytic activity in T-cell populations isolated directly from the draining lymph nodes (FIG. 12). Significantly, groups receiving Flt3-ligand pretreatment had consistently higher immune responses at day 5 and day 9 regardless of the adjuvant, i.e., IFA, Alum or Quil-A (FIGS. 10B, 10E, 10C and 10F, respectively). This data demonstrates that Flt3-ligand immunization protocols enhance immune responses to antigens that are formulated in a variety of adjuvants and that Flt3-ligand immunization protocols enhance immune responses over immunizing with adjuvant/antigen alone. Furthermore, mice receiving Flt3-ligand pretreatment and vaccinated with OT-I & II peptides in Alum or Quil-A showed a higher percentage of CD4+ transgenic T-cells at day 9, which suggest that Flt3-ligand immunization protocols maintain a heightened immune response for a longer period of time (FIG. 11F). Functional studies showed that CD4+ transgenic T cells from mice receiving Flt3-ligand pretreatment and vaccinated with OT-I & II peptides in IFA, Alum or Quil-A exhibited enhanced antigen-specific induction of IFNγ production over mice that did not receive Flt3-ligand pretreatment, and mice receiving Flt3-ligand pretreatment and vaccinated with OT-I & II peptides in IFA or Alum exhibited enhanced antigen-specific CD4+ T-cell proliferation over mice that did not receive Flt3-ligand pretreatment (FIG. 13). These data further confirm that Flt3-ligand immunization protocols result in heightened antigen-specific immune responses over standard vaccination techniques.

### Example 11

The following studies were performed to determine the effect of varying the number of days of Flt3-L pre-treatment on the generation of antigen-specific CTL post immunization. Six groups of mice were immunized to determine whether 10 days of FL treatment were necessary to achieve CTL (CD8+) expansion.

Approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with 25 ug each of the OT-1 and OT-II peptides emulsified in Incomplete Freund's Adjuvant (IFA—Difco/Becton Dickinson, Franklin Lakes, N.J.).

Group 1 received 10 ug Flt3-ligand s.c. per day at the nape of the neck for 10 consecutive days and immunized on day 11 with OT-I and OT-II peptides formulated in IFA. Group 2 received 10 ug Flt3-ligand s.c. per day at the nape of the neck for 8 consecutive days and immunized on day 11 with OT-I and OT-II peptides formulated in IFA. Group 3 received 10 ug Flt3-ligand s.c. per day at the nape of the neck for 6 consecutive days and immunized on day 11 with OT-I and OT-II peptides formulated in IFA. Group 4 received 10 ug Flt3-ligand s.c. per day at the nape of the neck for 4 consecutive days and immunized on day 11 with OT-I and OT-II peptides formulated in IFA. Group 5 was immunized on day 11 with OT-I and OT-Il peptides formulated in IFA. Group 6 received 10 ug Flt3-ligand s.c. per day at the nape of the neck for ten consecutive days and immunized on day 11 with IFA.

TABLE 15

| Grp | | Animals | Animals |
|---|---|---|---|
| # | Treatment Groups | per group | per time pt. |
| 1 | 10 days FL + IFA + p | 3 | 3 |
| 2 | 8 days FL + IFA + p | 3 | 3 |
| 3 | 6 days FL + IFA + p | 3 | 3 |
| 4 | 4 days FL + IFA + p | 3 | 3 |
| 5 | IFA + p | 3 | 3 |
| 6 | FL (10 days) + IFA − p | 3 | 3 |

The DLN were harvested from each group (of 3 mice) on day 6 post-immunization. The number of OTI CD8 T cells were quantified, and tested in a CTL assay. CTL activity was measured in a standard ⁵¹Cr-release assay. C1498 (H-2^{b}) target cells were pulsed with 50 μCi ⁵¹Cr (Amersham Biosciences, Piscataway, N.J.) per 1×10⁶ cells, in complete RPMI medium, for 1 hour at 37° C. with or without 1 μM OTI peptide (SIINFEKL, Immunex). Labeled target cells were washed four times and 1×10⁴ cells were added to serial titrations of DLN cells (effectors). Effector: target ratios ranged from 100:1 to 0.78:1. Assays were performed in complete RPMI medium containing RPMI-1640 (JRH Biosciences, Lenexa, Kans.) supplemented with 10% heat-inactivated fetal bovine serum (Gibco Invitrogen, Carlsbad, Calif.), 100 μM MEM non-essential amino acids (Gibco), 1 mM MEM sodium pyruvate (Gibco), 55 μM 2-mercaptoethanol (Gibco), 50 U/ml penicillin (Calbiochem, San Diego, Calif.), 50 μg/ml streptomycin (Mediatech, Herndon, Va.), and 2 mM L-glutamine (JRH Biosciences). After a 6-hour incubation at 37° C., 25 μl of cell-free supernatant was removed and transferred to a Packard LumaPlate (Packard BioScience, Meriden, Conn.). Plates were read for 60 seconds per well on the Packard TopCount (Hewlett Packard, Palto Alto, Calif.). Spontaneous and maximum chromium release were determined by the addition of assay medium or 0.1% Triton X-100 (Pierce Chemical Company, Rockford, Ill.) to target cells, respectively. Percent specific lysis was calculated as 100×(experimental release cpm−spontaneous release cpm)/(maximum release cpm−spontaneous release cpm).

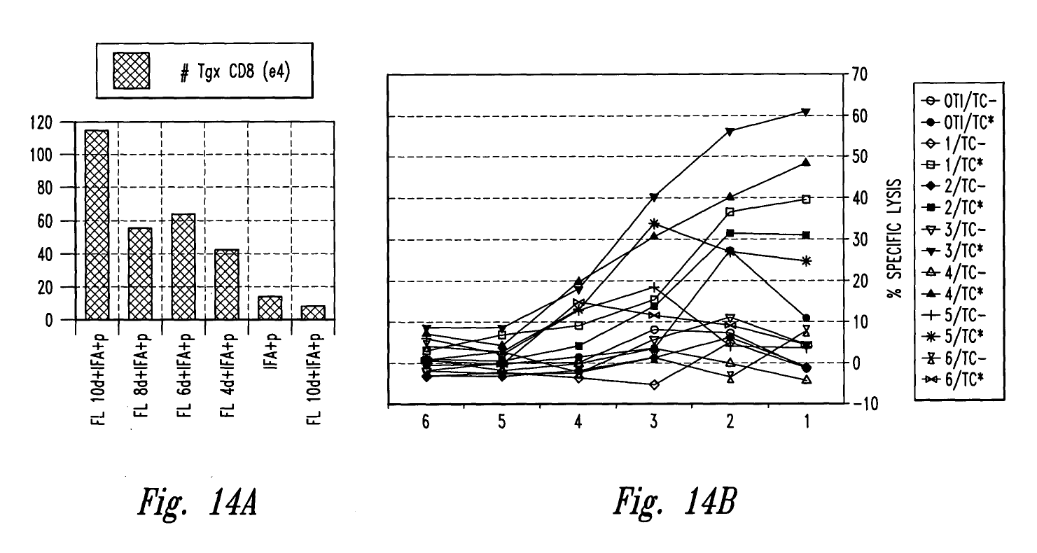

As shown in FIGS. 14A and 14B, OTI CD8 cell expansion was most noted in group 1, but expansion was also noted in groups 2-4, and to a lesser degree gp 5. Ex Vivo CTL activity was noted in Gps 1-5. These results show that ten days pre-treatment with Flt3-ligand was most effective at inducing expansion of OTI CD8 cells post immunization, but 8, 6 and 4 days pre-treatment were also effective compared to the control groups. Therefore, Flt3-ligand may be administered over a range of days (1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or more, as well as any combination of alternating days, such as , but not limited to every 2nd, 3rd, 4th, 5th, 6th, 7th days or more) in the Flt3-ligand immunization protocols described herein.

### Example 12

These studies show that antigen-specific CTL expansion occurs after immunization with whole ovalbumin protein, as compared to immunization with immunodominant peptides. As described in Examples 8 and 9, approximately two million splenocyte and lymph node cells from OT-I and OT-II transgenic mice were transferred into Ly5.1 congenic mice (equivalent to approx. 4×10⁵ CD4+ T and CD8+ T cells). Approximately twenty-four hours post transplantation, the mice were immunized subcuntaneously (s.c.) with 25 ug each of the OT-1 and OT-II peptides emulsified in Incomplete Freund's Adjuvant (IFA—Difco/Becton Dickinson, Franklin Lakes, N.J.). Six groups of mice were immunized as described below. Three mice were sacrificed on days 2, 6 and 9 post immunization. The DLN were harvested and OTI CD8 cells were quantified and tested in an ex vivo CTL assay described above in Example 11).

TABLE 16

| Grp | | Animals | Animals |
|---|---|---|---|
| # | Treatment Groups | per group | per time pt. |
| 1 | FL + IFA − peptides | 9 | 3 |
| 2 | FL + IFA + OT1 | 9 | 3 |
| | peptide plus OTII peptide | | |
| 3 | Saline + OVA in IFA (100 ug OVA) | 9 | 3 |
| 4 | FL + OVA in saline (100 ug OVA) | 9 | 3 |
| 5 | FL + OVA in IFA (100 ug OVA) | 9 | 3 |
| 6 | FL + OVA in IFA (25 ug OVA) | 9 | 3 |

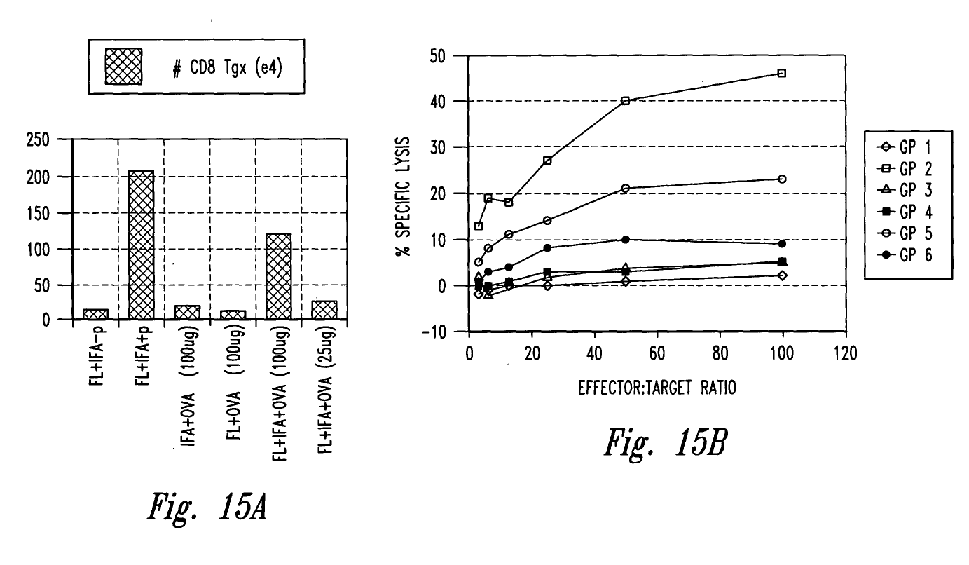

At the 6 day harvest, OTI CD8+ CTL cell expansion had occurred in mice immunized with peptides in IFA, and also in mice immunized with 100 ug OVA protein in IFA post FL treatment. As shown in FIGS. 15A and 15B, these experiments demontrate that Flt3-L pretreatment followed by immunization with antigen in IFA adjuvant is effective for use with both peptide and protein antigens. Notably, the OVA protein used for immunization was analyzed and shown to contain no free peptide. Therefore, Flt3-Ligand immunization protocols described herein may be used with a wide spectrum of antigens, as described in detail above.

### Example 13

The following studies demonstrate that treatment with Interleukin-15 post immunization augments antigen-specific effector CTL expansion.

Mice were treated with FL as described above for 10 days preceding immunization (see for example, Examples 8 and 9), in short, 10 ug Flt3-ligand s.c. per day at the nape of the neck for ten consecutive days and immunized on day 11 with IFA. Approximately two million splenocyte and lymph node cells from OT-I and OT-II transgenic mice were transferred into Ly.1 congenic mice on day 10 (equivalent to approx. 4×10⁵ CD4+ T and CD8+ T cells). Approximately twenty-four hours post transplantation, the mice were immunized subcuntaneously (s.c.) with 25 ug each of the OT-1 and OT-II peptides emulsified in Incomplete Freund's Adjuvant (IFA—Difco/Becton Dickinson, Franklin Lakes, N.J.). Animals were immunized on day 11 with peptides formulate in either saline or IFA. IL-15 or mouse serum albumin (MSA) control were delivered i.p at 10 ug/injection on days 3, 4, 5 and 6 (counting day of immunization as day 0).

TABLE 17

| | | | | | Animals |
|---|---|---|---|---|---|
| Grp | Recipient | Immunization | Treatment | Animals | per |
| # | Pre-imm | (Adjuvant) | Post-imm | per group | time pt. |
| 1 | FL | IFA + peptides | — | 9 | 3 |
| 2 | — | Saline + peptides | MSA | 6 | 3 |
| 3 | FL | Saline + peptides | MSA | 6 | 3 |
| 4 | — | IFA + peptides | MSA | 6 | 3 |
| 5 | — | Saline + peptides | IL-15 | 6 | 3 |
| 6 | FL | Saline + peptides | IL-15 | 6 | 3 |
| 7 | — | IFA + peptides | IL-15 | 6 | 3 |

Draining lymph nodes from 3 mice/group were harvested on days 5 and 9 post immunization, except group 1 which was harvested days 2, 5 and 9. OTI CD8 cells were quantified and CTL activity assayed in an ex vivo CTL assay.

CTL activity was measured in a standard ⁵¹Cr-release assay. C1498 (H-2^{b}) target cells were pulsed with 50 μCi ⁵¹Cr (Amersham Biosciences, Piscataway, N.J.) per 1×10⁶ cells, in complete RPMI medium, for 1 hour at 37° C. with or without 1 μM OTI peptide (SIINFEKL, Immunex). Labeled target cells were washed four times and 1×10⁴ cells were added to serial titrations of DLN cells (effectors). Effector: target ratios ranged from 100:1 to 0.78:1. Assays were performed in complete RPMI medium containing RPMI-1640 (JRH Biosciences, Lenexa, Kan.) supplemented with 10% heat-inactivated fetal bovine serum (Gibco Invitrogen, Carlsbad, Calif.), 100 μM MEM non-essential amino acids (Gibco), 1 mM MEM sodium pyruvate (Gibco), 55 μM 2-mercaptoethanol (Gibco), 50 U/ml penicillin (Calbiochem, San Diego, Calif.), 50 μg/ml streptomycin (Mediatech, Herndon, Va.), and 2 mM L-glutamine (JRH Biosciences). After a 6-hour incubation at 37° C., 25 μl of cell-free supernatant was removed and transferred to a Packard LumaPlate (Packard BioScience, Meriden, Conn.). Plates were read for 60 seconds per well on the Packard TopCount (Hewlett Packard, Palto Alto, Calif.). Spontaneous and maximum chromium release were determined by the addition of assay medium or 0.1% Triton X-100 (Pierce Chemical Company, Rockford, Ill.) to target cells, respectively. Percent specific lysis was calculated as 100×(experimental release cpm−spontaneous release cpm)/(maximum release cpm−spontaneous release cpm).

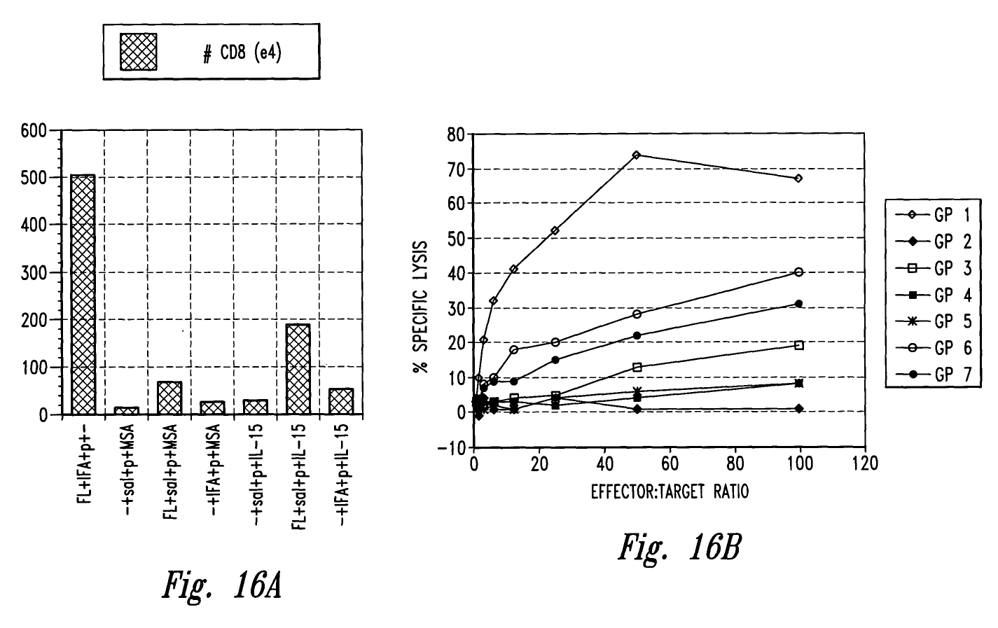

As shown in FIGS. 16A and 16B, these results demonstrate that IL-15 given post immunization augmented OTI CD8 expansion and function when mice were pre-treated with Flt3-ligand and immunized with peptides in saline (compare groups 3 and 6). A lesser effect was noted when IL-15 was administered post immunization with peptides fromulated in IFA (after no Flt3-ligand treatment), i.e., compare groups 4 and 7. Therefore, the utility and efficacy of administering auxilliary molecules in Flt3-ligand immunization protocols has been demonstrated.

### Example 14

The following studies demonstrate that administration of anti-4-1BB agonistic antibody or IL-15 post immunization augments the generation of a pool of memory CD8+ antigen-specific T cells.

Mice were treated with FL as described above for 10 days preceding immunization (see for example, Examples 8 and 9), in short, 10 ug Flt3-ligand s.c. per day at the nape of the neck for ten consecutive days and immunized on day 11 with IFA. Approximately two million splenocyte and lymph node cells from OT-I and OT-II transgenic mice were transferred into Ly5.1 congenic mice on day 10 (equivalent to approx. 4×10⁵ CD4+ T and CD8+ T cells). Approximately twenty-four hours post transplantation, the mice were immunized subcuntaneously (s.c.) with 25 ug each of the OT-1 and OT-II peptides emulsified in Incomplete Freund's Adjuvant (IFA—Difco/Becton Dickinson, Franklin Lakes, N.J.). All animals were immunized with OTI plus OTII peptides in saline (gps 1-3) or IFA (gps 4-9).

Within each adjuvant group, a group received rat IgG or agonistic anti-4-1BB antibody on day 3 and day 6 post immunization (100 ug/injection, i.p.) and a third group received recombinant human IL-15 (commercially available, see for example R&D Systems, Minneapolis, Minn.) on days 3 through 6 at 10 ug/injection, i.p.

TABLE 18

| Grp | | | Animals | Animals |
|---|---|---|---|---|
| # | Treatment Groups | | per group | per time pt. |
| 1 | FL + peptides | rIgG | 8 | 8 |
| 2 | FL + peptides | IL-15 | 8 | 8 |
| 3 | FL + peptides | Anti-4-1BB | 8 | 8 |
| 4 | IFA + peptides | rIgG | 8 | 8 |
| 5 | IFA + peptides | IL-15 | 8 | 8 |
| 6 | IFA + peptides | Anti-4-1BB | 8 | 8 |
| 7 | FL + IFA + peptides | rIgG | 8 | 8 |
| 8 | FL + IFA + peptides | IL-15 | 8 | 8 |
| 9 | FL + IFA + peptides | Anti-4-1BB | 8 | 8 |

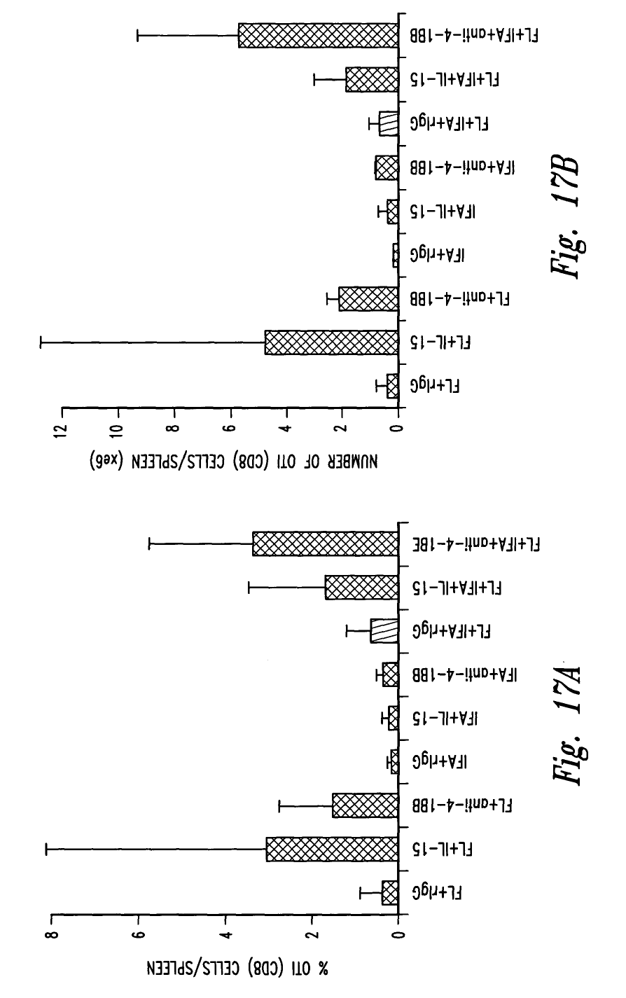

Spleens were harvested from 3 mice at weeks 9, 17 and 2 mice at week 18 post immunization. OTI CD8+ T-cells were quantified, as described in previous Examples, from individual spleens by flow cytometry. As shown in FIGS. 17A and 17B, treatment with IL-15 or anti-4-1BB immediately post immunization augments the size of the memory T cell pool. This effect was noted after immunization with Flt3-ligand plus peptides formulated in saline (gps 1-3) or Flt3-ligand plus peptides formulated in IFA (gps 7-9), but not after immunization in the absence of Flt3-ligand (gps 4-6).

### Example 15

Flt3-L may be used in the treatment of allergies. The Flt3-L immunization protocols described throughout this specification have direct utility in the allergen-specific immunotherapy of allergies. Allergen-specific immunotherapy is defined as the administration of increasing doses of an allergen vaccine to a subject having one or more allergies in order to reach a dose effective to improve symptoms associated with subsequent exposure to the causative allergen. Immunotherapy of allergies includes Flt3-L immunization protocols and allergen-specific immunotherapy of allergies and/or desensitation therapy that have been modified to include administration of F13-L.

Any suitable in vivo and in vitro techniques employed in the art of allergological diagnosis may be used to diagnose a subject, including more conventional tests such as but not limited to intradermal Serial Endpoint Testing (SET), radioallergosorbent assay (RAST), RAST Spot Test, Histamine Radioensymatic Assays, in vitro IgE and IgG assays, spontaneous synthesis assays, as well as other assays known in the art.

Allergy vaccines are well-known in the art, and can generally be defined as comprising at least one allergen and any suitable carrier, diluent, exipient, stabilizer and optional adjuvant. An allergen is defined herein as any art-recognized allergen; modified allergens (modified by such methods as, but not limited to, urea, PEG/PVA, deglycosylation, polysaccharides and/or photooxidation); allergoids (modified by such methods as, but not limited to, glutaraldehyde and/or formaldehyde treatment with or without tyrosine absorbtion); monovalent allogenic extracts; allergen polymers; conjugated allergens; allergen-muramylpeptides of allergens; allergen mycoloilmuramylpeptide conjugates; allergen-pullulan compounds; conjugates of allergen and hapten(s); conjugates of allergen, hapten(s) and hydrophilic polymers; urea denaturod antigens; recombinant allergens; mutagenized recombinant allergens; genetically-engineered allergens such as hypoallergens and/or hypoallergenic derivatives (e.g., molecules with reduced IgE-binding epitopes but preserving T-cell epitopes and epitopes for the induction of IgG antibodies that may serve as blocking antibodies);

The allergy vaccine and Flt3-L may be adminstered in any efficacious manner and route described herein. Dosing and administration of the allergen vaccine and Flt3-L may be determined by qualified physicians.

Flt3-L immunization protocols can be used in allergy immunotherapy for any treatable allergies, which includes, but is not limited to: insect allergies and insect bites and/or stings (dust mites, ants, spiders, flies, bees, wasps, mosquitoes, gnats and the like); animal allergies (fur, dander, excrement, etc. of domesticated and wild, such as, but not limited to: dogs, cats, birds, rodents, cows, sheep, horse, pigs, goats and the like); allergic bronchitis, balsam allergy; candida allergy; carpet and/or fabric allergy; food allergies or sensitivities; allergies to metals; colophony allergy; disinfectant allergy; fertilizer allergy; formaldehyde allergy; gas allergy; glue allergy; allogeneic serum allergy; jewelry allergy; mercapto allergy; mold and/or mildew allergy; paint allergy; paper allergy; parabens allergy; perfume allergy; pesticide allergy; plastic allergy; shampoo allergy; soap allergy; thiuram allergy; tobacco allergy; wheat allergy; yeast allergy; allergic dermatitis; allergic rhinitis; aspirin sensitivity; asthma; atopic dermatitis; contact dermatitis; cosmetic allergy; cows milk allergy; dermatitis; dust allergy; pollen allergy; eczema; grass allergy; salicylate sensitivity and the like.

As described in detail above, Flt3-L may be administered to a subject prior to, concurrent with and/or subsequent to the administration of a allergy vaccine and optional auxiliary molecules. In one embodiment, Flt3-L is administered to the subject once a day, everyday every 2^{nd}, 3^{rd}, 4^{th}, 5^{th}, 6^{th} or 7^{th} day for 1, 2, 3, 4, 5, 6, 7, 8, 9, 10, 11, 12, 13, 14, 15, 16, 17, 18, 19 or 20 consecutive days prior to, concurrent with and/or subsequent to vaccination. Of course, all the embodiments of Flt3-L immunization protocols desribed above may be adapted to the treatment of allergies. Furthermore, existing allergy immunization regimens known in the art may be modified to include administration of Flt3-L.

In one embodiment, Flt3-ligand immunization protocols relate to a method of treating allgeries in a subject having one or more allergies, comprising the steps of:

(a) administering Flt3-ligand to the subject;

(b) optionally administering an auxiliary molecule; and,

(c) administering an allergy vaccine to the subject, wherein Flt3-ligand is administered prior to, concurrent with and/or subsequent to administration of the vaccine, and wherein the auxiliary molecule is administered prior to, concurrent with and/or subsequent to administration of the vaccine, and wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

As described above, Flt3-L expands hematopoietic stem and progenitor cells as well as various types of immune cells, especially dendritic cells. Furthermore, Flt3-L has the capacity to expand Th1-type dendritic cells. Therefore, Flt3-L immunization protocols in allergy immunotherapy can drive the subject's immune response from a typical Th2 respone to a Th1-type response. This shift in cytokine profile and immune response will drive IgG production over IgE production, decrease circulating levels of IL-4, decrease recruitment and activation of eosinophils, as well as decrease proliferation of mast cells. As a result, subsequent allergen exposure does not provoke an allergic reaction.

The efficacy of allergen-specific immunotherapy using a Flt3-L immunization protocol may be evaluated by standard methods and techniques known in the art, such as but not limited to measurement of allergen-specific IgG and IgE antibodies from the patient. Patients undergoing allergen-specific immunotherapy in a Flt3-L immuniztion protocol may also be treated in combination with one or more conventional therapies, such as but no limited to, antihistamines, decongestants, steroids, analgesics, cough suppressants, and the like.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

| SEQ ID NO: | Description of sequence |
|---|---|
| 1 | Amino acid sequence for human Flt3-ligand |
| 2 | Amino acid sequence for murine Flt3-ligand |
| 3 | Peptide sequence for TO-I transgenics |
| 4 | Peptide sequence for TO-II transgenics |

What is claimed is:

1. A method of immunizing a subject, comprising the steps of:
(a) administering Flt3-ligand to a subject;
(b) optionally administering an auxiliary molecule; and,
(c) administering a vaccine to the subject, wherein the vaccine comprises an antigen and an adjuvant.

2. The method of claim 1, wherein Flt3-ligand is administered prior to, concurrent with and/or subsequent to administration of the vaccine.

3. The method of claim 1, wherein the auxiliary molecule is administered prior to, concurrent with and/or subsequent to administration of the vaccine.

4. The method of claim 1, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

5. The method of claim 1, wherein the adjuvant is selected from the group consisting of ADJUMER™ (polyphosphazene); aluminum phosphate gel; algal glucans; algammulin; aluminum hydroxide gel (alum); high protein adsorbency aluminum hydroxide gel; low viscosity aluminum hydroxide gel; AF or SPT (emulsion of squalane (5%), Tween 80(0.2%), Pluronic L121(1.25%), phosphate-buffered saline pH 7.4); AVRIDE™ (propanediamine); BAY R1005™ ((N-(2-Deoxy-2-L-leucylamino-b-D-glucopyranosyl)-N-octadecyldodecanoylamide hydroacetate); CALCITRIOL™ (1α, 25-dihydroxyvitamin D3); calcium phosphate gel; CAP™ (calcium phosphate nanoparticles); cholera holotoxin, cholera toxin A1-protein A-D fragment fusion protein, cholera toxin B subunit; CRL 1005 (Block Copolymer P1205); cytokine containing liposomes; DDA (dimethyldioctadecylammonium bromide); DHEA (dehydroepiandrosterone); DMPC (dimyristoyl phosphatidylcholine); DMPG (dimyristoyl phosphatidylglycerol); DOC/Alum Complex (deoxycholic Acid Sodium Salt); Freund's Complete Adjuvant; Freund's Incomplete Adjuvant; Gamma Inulin; Gerbu Adjuvant (mixture of: i) N-Acetylglucosaminyl-(P1-4)-N-acetylmuramyl-L-alanyl-D-glutamine (GMDP), ii) Dirnethyl dioctadecylammonium. chloride (DDA), iii) Zinc L-proline salt complex (ZnPro-8); GM-CSF; GMDP (N-acetylglucosaminyl-(b1-4)-N-acetylmuramyl-L-alanyl-D-isoglutamine); Imiquimod (1-(2-methypropyl)-IH-imidazo[4,5-c]quinolin-4-amine); ImmTher™ (N-acetylglucosaminyl-N-acetyhnuramyl-L-Ala-D-isoGlu-L-Ala-glycerol dipalmitate); DRVs (Immunoliposomes prepared from Dehydration-Rehyrdation Vesicles); Interferon-γ; Interleukin-1β; Interleukin-2; Interleukin-7; Interleukin-12; ISCOMS™ (Immune Stimulating Complexes); ISCOPREP 7.0.3. ™; Liposomes; LOXORIBINE™ (7-allyl-8-oxoguanosine); LT Oral Adjuvant™ (E. coli labile enterotoxin protoxin); Microspheres and Microparticles of any composition; MF59™; (squalene.water emulsion); MONTANIDE ISA 51™ (purified Incomplete Freund's Adjuvant); MONTANIDE ISA 720™ (metabolizable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxy-phosphoryloxy)) ethylamide, mono sodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glycerol dipalmitoyl); NAGO (Neuraminidase-galactose oxidase); Nanospheres or Nanoparticles of any composition; NISVs (Non-Ionic Surfactant Vesicles); PLEURAN™ (β-glucan); PLGA, PGA and PLA (homo-and co-polymers of lactic and glycolic acid; micro-/nanospheres); PLURONIC L121™; PMMA (polymethyl methacrylate); PODDS™ (oroteinoid microspheres); Polyethylene carbamate derivatives; Poly rA:Poly rU (Poly-adenylic acid-poly-uridylic acid complex); Polysorbate 80 (Tween 80); Protein Cochleates (Avanti Polar Lipids, Inc., Alabaster, Ala.); STIMULON™ (QS-21); Quil-A (Quil-A saponin); S-28463 (4-Amino-otec,-dimethyl-2-ethoxymethyl-lH-imidazo[4,5-c]quinoline-1-ethanol); SAF-1™ (Syntex Adjuvant Formulation); Sendai proteoliposomes and Sendai-containing lipid matrices; Span-85 (sorbitan trioleate); Specol (emulstion of Marcol 52, Span 85 and Tween 85); Squalene or Robane® (2,6,10,15,19,23-hexamethyltetracosane and 2,6,10,15,19,23-hexamethyl-2,6,10,14,18,22 tetracosahexaene); Stearyl Tyrosine (Octadecyl tyrosine hydrochloride); Theramide® (N-acetylglucosaminyl-N-acetylinuramyl-L-Ala-D-isoGlu-L-Ala-dipalmitoxy propylamide); Theronyl-MDP (Termurtide™ or [thr 1]-MDP; N-acetyl muramyl-L-threonyl-D-isoglutamine); Ty Particles (Ty-VLPs or virus like particles); Walter Reed Liposomes

6. The method of claim 1, wherein the antigen is a cancer antigen.

7. The method of claim 6, wherein the cancer antigen is selected from the group consisting of Melanoma-Melanocyte Differentiation Antigens (MART-1/Melan A; gp100/pmel-17; Tyrosinase; Tyronsinase Related Protein-1; Tyronsinase Related Protein-2; Melanocyte-Stimulating Hormone Receptor); Cancer-Testes Antigens (MAGE-1; MAGE-2; MAGE-3; MAGE-12, BAGE; CAGE, NYESO-1); Mutated Antigens (β-catenin; MUM-1; CDK-4; Caspase-8; KIA 0205; HLA-A2-R1701); and Non-Mutated Shared Antigens Overexpressed on Cancers (α-Fetoprotein; Telomerase Catalytic Protein; G-250; MUC-1; Carcinoembryonic antigen; p53; Her-2/neu), epitopes from Non-Mutated Proteins (gp100; MAGE-1; MAGE-3; Tyrosinase; NY-ESO-1) and epitopes from Mutated Proteins (Triosephosphate isomerase; CDC-27; LDLR-FUT).

8. The method of claim 1, wherein the antigen is a viral antigen.

9. The method of claim 8, wherein the viral antigen is selected from the group consisting of Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunga viruses, phleboviruses and Nairo viruses); Arena viridae (hemorrhagic fever viruses); Reoviridae (e.g., reoviruses, orbiviuises and rotaviruses); Birnaviridae; Hepadnaviridae (Hepatitis B virus); Parvoviridae (parvovirusies); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxviridae (variola viruses, vaccinia viruses, pox viruses); and Iridoviridae (e.g., African swine fever virus); viral agents of non-A, non-B hepatitis; Norwalk and related viruses, and astroviruses).

10. The method of claim 1, wherein the antigen is a bacterial antigen.

11. The method of claim 10, wherein the bacterial antigen is selected from the group consisting of Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

12. The method of claim 1, wherein the antigen is from an infectious unicellular organism.

13. The method of claim 12, wherein the antigen is selected from the group consisting of schistosomes; trypanosomes; Leishmania species; filarial nematodes; trichomoniasis; sarcosporidiasis; Taenia saginata, Taenia solium, Cryptococcus neoformans, Apergillus fumigatus, Histoplasma capsulatum, Coccidiodes immitis, trichinelosis, Blastomyces dermatitidis, Chlamydia trachomatis, Candida albicans, Plasmodium falciparum, Plasmodium vivax, Plasmodium malariae, and Toxoplasma gondii.

14. A method of treating cancer in a subject having cancer, comprising the steps of:
(a) administering Flt3-ligand to a subject;
(b) optionally administering an auxiliary molecule; and,
(c) administering a vaccine to the subject, wherein the vaccine comprises a cancer antigen and an adjuvant.

15. The method of claim 14, wherein Flt3-ligand is administered prior to, concurrent with and/or subsequent to administration of the vaccine.

16. The method of claim 14, wherein the auxiliary molecule is administered prior to, concurrent with and/or subsequent to administration of the vaccine.

17. The method of claim 14, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-IBB binding proteins, OX-40 binding proteins, CpG sequences, and combinations thereof.

18. A method of preventing and/or treating viral infection in a subject, comprising the steps of:
(a) administering Flt3-ligand to a subject;
(b) optionally administering an auxiliary molecule; and,
(c) administering a vaccine to the subject, wherein the vaccine comprises a viral antigen and an adjuvant.

19. The method of claim 18, wherein Flt3-ligand is administered prior to, concurrent with and/or subsequent to administration of the vaccine.

20. The method of claim 18, wherein the auxiliary molecule is administered prior to, concurrent with and/or subsequent to administration of the vaccine.

21. The method of claim 18, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

22. A method of preventing and/or treating bacterial infection in a subject, comprising the steps of:
(a) administering Flt3-ligand to a subject;
(b) optionally administering an auxiliary molecule; and,
(c) administering a vaccine to the subject, wherein the vaccine comprises a bacterial antigen and an adjuvant.

23. The method of claim 22, wherein Flt3-ligand is administered prior to, concurrent with and/or subsequent to administration of the vaccine.

24. The method of claim 22, wherein the auxiliary molecule is administered prior to, concurrent with and/or subsequent to administration of the vaccine.

25. The method of claim 22, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

26. A method of enhancing an immune response to an antigen in a subject, comprising the steps of:
(a) administering Flt3-ligand to a subject;
(b) optionally administering an auxiliary molecule; and,
(c) administering a vaccine to the subject, wherein the vaccine comprises an antigen and an adjuvant.

27. The method of claim 26, wherein Flt3-ligand is administered prior to, concurrent with and/or subsequent to administration of the vaccine.

28. The method of claim 26, wherein the auxiliary molecule is administered prior to, concurrent with and/or subsequent to administration of the vaccine.

29. The method of claim 26, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

30. A method of enhancing an antigen-specific cytotoxic T-cell immune response to an antigen in a subject, comprising the steps of:
(a) administering Flt3-ligand to a subject;
(b) optionally administering an auxiliary molecule; and,
(c) administering a vaccine to the subject, wherein the vaccine comprises an antigen and an adjuvant.

31. The method of claim 30, wherein Flt3-ligand is administered prior to, concurrent with and/or subsequent to administration of the vaccine.

32. The method of claim 30, wherein the auxiliary molecule is administered prior to, concurrent with and/or subsequent to administration of the vaccine.

33. The method of claim 31, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-IBB binding proteins, OX-40 binding proteins, CpG sequences, and combinations thereof.

34. A method of enhancing an antigen-specific T-helper immune response to an antigen in a subject, comprising the steps of:
(a) administering Flt3-ligand to a subject;
(b) optionally administering an auxiliary molecule; and,
(c) administering a vaccine to the subject, wherein the vaccine comprises an antigen and an adjuvant.

35. The method of claim 34, wherein Flt3-ligand is administered prior to, concurrent with and/or subsequent to administration of the vaccine.

36. The method of claim 34, wherein the auxiliary molecule is administered prior to, concurrent with and/or subsequent to administration of the vaccine.

37. The method of claim 34, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

38. A method of evaluating the immune responses to an antigen in a subject, comprising the steps of:
(a) administering Flt3-ligand to a subject;
(b) optionally administering an auxiliary molecule;
(c) administering an antigen to the subject, wherein the antigen may optionally be formulated with an adjuvant; and,
(d) evaluating the subject's immune responses to the antigen.

39. The method of claim 38, wherein Flt3-ligand is administered prior to, concurrent with and/or subsequent to administration of the vaccine.

40. The method of claim 38, wherein the auxiliary molecule is administered prior to, concurrent with and/or subsequent to administration of the vaccine.

41. The method of claim 38, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.

42. A method of treating allgeries in a subject having one or more allergies, comprising the steps of:
(a) administering Flt3-ligand to the subject;
(b) optionally administering an auxiliary molecule; and,
(c) administering an allergy vaccine to the subject.

43. The method of claim 42, wherein Flt3-ligand is administered prior to, concurrent with and/or subsequent to administration of the allergy vaccine.

44. The method of claim 42, wherein the auxiliary molecule is administered prior to, concurrent with and/or subsequent to administration of the allergy vaccine.

45. The method of claim 42, wherein the auxiliary molecule is selected from the group consisting of Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF- [symbol] , TGF-β, soluble CD40 ligand, CD40-binding proteins, soluble CD83, 4-1BB binding proteins, OX-40 binding proteins, CpG sequences, and combinations thereof.
